(19) United States
(12) Patent Application Publication
(10) Pub. No.: US 2003/0220371 A1
(43) Pub. Date: Nov. 27, 2003

(54) Compounds and methods

(76) Inventors: Lara S. Kallander, King of Prussia, PA (US); Scott Thompson, King of Prussia, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/257,307

(22) PCT Filed: Apr. 12, 2001
(86) PCT No.: PCT/US01/11979

(51) Int. Cl. A61K 31/4439; A61K 31/4192

(52) U.S. Cl. 514/340; 514/359

(57) ABSTRACT

Compounds of this invention are non-peptide, reversible inhibitors of type 2 methionine aminopeptidase, useful in treating conditions mediated by angiogenesis, such as cancer, haemangioma, proliferative retinophathy, rheumatoid arthritis, atherosclerotic neovascularization, psoriasis, ocular neovascularization and obesity.

### FIELD OF THE INVENTION

Compounds of this invention are non-peptide, reversible inhibitors of type 2 methionine aminopeptidase, useful in treating conditions mediated by angiogenesis, such as cancer, haemangioma, proliferative retinopathy, rheumatoid arthritis, atherosclerotic neovascularization, psoriasis, ocular neovascularization and obesity.

### BACKGROUND OF THE INVENTION

In 1974, Folkman proposed that for tumors to grow beyond a critical size and to spread to form metastases, they must recruit endothelial cells from the surrounding stroma to form their own endogenous microcirculation in a process termed angiogenesis (Folkman J. (1974) Adv Cancer Res. 19; 331). The new blood vessels induced by tumor cells as their life-line of oxygen and nutrients also provide exits for cancer cells to spread to other parts of the body. Inhibition of this process has been shown to effectively stop the proliferation and metastasis of solid tumors. A drug that specifically inhibits this process is known as an angiogenesis inhibitor.

Having emerged as a promising new strategy for the treatment of cancer, the anti-angiogenesis therapy (“indirect attack”) has several advantages over the “direct attack” strategies. All the “direct attack” approaches such as using DNA damaging drugs, antimetabolites, attacking the RAS pathway, restoring p53, activating death programs, using aggressive T-cells, injecting monoclonal antibodies and inhibiting telomerase, etc., inevitably result in the selection of resistant tumor cells. Targeting the endothelial compartment of tumors as in the “indirect attack”, however, should avoid the resistance problem because endothelial cells do not exhibit the same degree of genomic instability as tumor cells. Moreover, anti-angiogenic therapy generally has low toxicity due to the fact that normal endothelial cells are relatively quiescent in the body and exhibit an extremely long turnover. Finally since the “indirect attack” and “direct attack” target different cell types, there is a great potential for a more effective combination therapy.

More than 300 angiogenesis inhibitors have been discovered, of which about 31 agents are currently being tested in human trials in treatment of cancers (Thompson, et al., (1999) J Pathol 187, 503). TNP-470, a semisynthetic derivative of fumagillin of Aspergillus fuigatus, is among the most potent inhibitors of angiogenesis. It acts by directly inhibiting endothelial cell growth and migration in vitro and in vivo (lngber et al. (1990) Nature 348, 555). Fumagillin and TNP-470, have been shown to inhibit type 2 methionine aminopeptidase (hereinafter MetAP2) by irreversibly modifying its active site. The biochemical activity of fumagillin analogs has been shown to correlate to their inhibitory effect on the proliferation of human umbillical vein endothelial cells (HUVEC). Although the mechanism of the selective action of fumagillin and related compounds on MetAP2-mediated endothelial cell cytostatic effect has not yet been established, possible roles of MetAP2 in cell proliferation have been suggested.

First, hMetAP-2-catalyzed cleavage of the initiator methionine of proteins could be essential for releasing many proteins that, after myristoylation, function as important signaling cellular factors involved in cell proliferation. Proteins known to be myristoylated include the src family tyrosine kinases, the small GTPase ARF, the HIV protein nef and the α subunit of heterotrimeric G proteins. A recently published study has shown that the myristoylation of nitric oxide synthase, a membrane protein involved in cell apoptosis, was blocked by fumagillin (Yoshida, et al. (1998) Cancer Res. 58(16), 3751). This is proposed to be an indirect outcome of inhibition of MetAP2-catalyzed release of the glycine-terminal myristoylation substrate. Alternatively, MetAP enzymes are known to be important to the stability of proteins in vivo according to the “N-end rule” which suggests increased stability of methionine-cleaved proteins relative to their N-terminal methionine precursors (Varshavslky, A (1996) Proc. Natl. Acad. Sci. U.S.A. 93, 12142). Inhibition of hMetAP2 could result in abnormal presence or absence of some cellular proteins critical to the cell cycle.

Methionine aminopeptidases (MetAP) are ubiquitously distributed in all living organisms. They catalyze the removal of the initiator methionine from newly translated polypeptides using divalent metal ions as cofactors. Two distantly related MetAP enzymes, type 1 and type 2, are found in eukaryotes, which at least in yeast, are both required for normal growth; whereas only one single MetAP is found in eubacteria (type 1) and archaebacteria (type 2). The N-terminal extension region distinguishes the methionine aminopeptidases in eukaryotes from those in procaryotes. A 64-amino acid sequence insertion (from residues 381 to 444 in hMetAP2) in the catalytic C-terminal domain distinguishes the MetAP-2 family from the MetAP-1 family. Despite the difference in the gene structure, all MetAP enzymes appear to share a highly conserved catalytic scaffold^{termed} “pita-bread” fold (Bazan, et al. (1994) Proc. Natl. Acad. Sci. U.S.A. 91, 2473), which contains six strictly conserved residues implicated in the coordination of the metal cofactors.

Mammalian type 2 methionine aminopeptidase has been identified as a bifunctional protein implicated by its ability to catalyze the cleavage of N-terminal methionine from nascent polypeptides (Bradshaw, et al (1998) Trends Biochem. Sci. 23, 263) and to associate with eukaryotic initiation factor 2α (eIF-2α) to prevent its phosphorylation (Ray, et al. (1992) Proc. Natl. Acad. Sci. U.S.A. 89, 539). Both the genes of human and rat MetAP2 were cloned and have shown 92% sequence identity (Wu., et al. (1993) J Biol. Chem. 268, 10796; Li, X. & Chang, Y.-H. (1996) Biochem. & Biophys. Res. Comm. 227, 152). The N-terminal extension in these enzymes is highly charged and consists of two basic polylysine blocks and one aspartic acid block, which has been speculated to be involved in the binding of eIF-2α (Gupta, et al. (1993) in Translational Regulation of Gene Expression 2 (Ilan, J., Ed.), pp. 405-431, Plenum Press, New York).

The anti-angiogenic compounds, fumagillin and its analogs, have been shown to specifically block the exo-aminopeptidase activity of hMetAP2 without interfering with the formation of the hMetAP2: eIF2α complex (Griffith, et al., (1997) Chem. Biol. 4, 461; Sin, et al. (1997) Proc. Natl. Acad. Sci. U.S.A. 94, 6099). Fumagillin and its analogs inactivate the enzymatic activity of hMetAP2 with a high specificity, which is underscored by the lack of effect of these compounds on the closely related type 1 methionine aminopeptidase (MetAP1) both in vitro and in vivo in yeast (Griffith, et al., (1997) Chem. Biol. 4, 461; Sin, et al. (1997) Proc. Natl. Acad. Sci. U.S.A. 94, 6099). The extremely high potency (IC50<1 nM) of these inhibitors appears to be due to the irreversible modification of the active site residue, His231, of hMetAP2 (Liu, et al. (1998) Science 282, 1324). Disturbance of MetAP2 activity in vivo impairs the normal growth of yeast (Griffith, et al., (1997) Chem. Biol. 4, 461; Sin, et al. (1997) Proc. Natl. Acad. Sci. U.S.A. 94, 6099; In-house data) as well as Drosophila (Cutforth & Gaul (1999) Mech. Dev. 82, 23). Most significantly, there appears to be a clear correlation between the inhibition effect of fumagillin related compounds against the enzymatic activity of hMetAP2 in vitro and the suppression effect of these compounds against tumor-induced angiogenesis in vivo (Griffith, et al., (1997) Chem. Biol. 4, 461).

Cancer is the second leading cause of death in the U.S., exceeded only by heart disease. Despite recent successes in therapy against some forms of neoplastic disease, other forms continue to be refractory to treatment. Thus, cancer remains a leading cause of death and morbidity in the United States and elsewhere (Bailar and Gornik (1997) N Engl J Med 336, 1569). Inhibition of hMetAP2 provides a promising mechanism for the development of novel anti-angiogenic agents in the treatment of cancers. It has now been discovered that compounds of formulae (I) and (IA) are effective inhibitors of hMetAP2, and thus would be useful in treating conditions mediated by hMetAP2.

### SUMMARY OF THE INVENTION

In one aspect, the present invention is to a novel compound of formula (I), or a pharmaceutically active salt or solvate thereof, and, further, its use in treating conditions mediated by angiogenesis, such as cancer, haemangioma, proliferative retinopathy, rheumatoid arthritis, atherosclerotic neovascularization, psoriasis, ocular neovascularization and obesity:

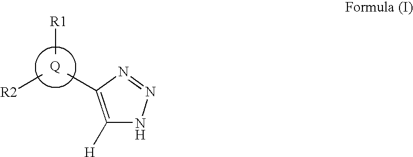

wherein:

Q is a 5- or 6-membered monocyclic ring optionally containing up to two heteroatoms selected from N, O, or S, or an 8- to 11-membered fused bicyclic ring optionally containing up to four heteroatoms selected from N, O, or S;

with the proviso that Q is substituted by up to eight of R¹; and further, if Q is phenyl (“Ph”), Q must be substituted by at least one of substituent R²;

R¹ is H—, Ph-C₀₋₆alkyl-, Het-C₀₋₆ alkyl-, C₁₋₆alkyl-, C₁₋₆alkoxy-, C₁₋₆mercaptyl-, Ph-C₀₋₆alkoxy-, Het-C₀₋₆alkoxy-, HO—, R⁴R⁵N—, Het-S—C₀₋₆alkyl-, Ph-S—C₀₋₆alkyl-, HO(CH₂)₁₋₆—, R⁴R⁵N(CH₂)₂₋₆—, R⁴R⁵N(CH₂)₂₋₆O—, R⁶CO₂(CH₂)₀₋₆—, R⁶CO₂(CH₂)₁₋₆O—, R⁶SO₂(CH₂)₁₋₆—, —CF₃, —OCF₃, or halogen, and Ph or Het are substituted with up to five of C₂₋₆alkyl-, C₁₋₆alkoxy-, R⁴R⁵N(CH₂)₁₋₆-, R⁴R⁵N(CH₂)₂₋₆O—, —CO₂R⁶, —CF₃ or, halogen;

R² is Ph-C₀₋₆alkyl-, Het-C₀₋₆alkyl-, C₅₋₆alkyl-, C₂₋₆alkoxy-, C₁₋₆mercaptyl-, Ph-C₀₋₆alkoxy-, Het-C₀₋₆alkoxy-, HO—, R⁴R⁵N—, Het-S—C₀₋₆alkyl-, Ph-S—C₀₋₆alkyl-, HO(CH₂)₁₋₆—, R⁴R⁵N(CH₂)₂₋₆—, R⁴R⁵N(CH₂)₂₋₆O—, R⁶CO₂(CH₂)₀₋₆—, R⁶CO₂(CH₂)₁₋₆O—, R⁶SO₂(CH₂)₁₋₆—, —CF₃ or —OCF₃, and Ph or Het are substituted with up to five of C₂₋₆alkyl-, C₁₋₆alkoxy-, R⁴R⁵N(CH₂)₁₋₆—, R⁴R⁵N(CH₂)₂₋₆O—, —CO₂R⁶, —CF₃ or, halogen;

provided that the compound of formula (I) is not [(6-(1H-1,2,3-triazol-4-yl)-2-napthalenyl)oxy]-acetic acid; [(6-(1H-1,2,3-triazol-4-yl)-2-napthalenyl)oxy]-acetic acid 1,1-dimethylethyl ester; 4-(1H-1,2,3-triazol-4-yl)-aniline; 2-chloro-4-(1H-1,2,3-triazol-4-yl)-aniline; 1-(4-fluorophenyl)-5-(1H-1,2,3-triazol-4-yl)-1H-indole; 2-(1H-1,2,3-triazol-4-yl)-pyridine; 3-(1H-1,2,3-triazol-4-yl)-pyridine; 4-(1H-1,2,3-triazol-4-yl)-phenol; 4-(2-napthyl)-1H-1,2,3-triazole; 4-[3-bromo-4-(trifluoromethoxy)phenyl]-1H-1,2,3-triazole; 4-(1H-1,2,3-triazol-4-yl)-morpholine; 5-methyl-2-(1H-1,2,3-triazol-4-yl)-1H-benzimidazole; 1-(1H-1,2,3-triazol-4-yl)-1H-benzotriazole; 5-methyl-2-(1H-1,2,3-triazol-4-yl)-1H-benzotriazole; or 3-(1H-1,2,3-triazol-4-yl)-piperidine; and

R⁴, R⁵, and R⁶ are independently selected from H—, C₂₋₆alkyl-, C₃₋₆alkenyl-, C₃₋₆alkynyl-, Ph-C₀₋₆alkyl-, Het-C₀₋₆alkyl-, or C₃₋₇cycloalkyl-C₀₋₆alkyl-.

In a second aspect, the present invention is to a method of treating conditions mediated by angiogenesis, such as cancer, haemangioma, proliferative retinopathy, rheumatoid arthritis, atherosclerotic neovascularization, psoriasis, ocular neovascularization and obesity by administering a compound of formula (IA), or a pharmaceutically acceptable salt or solvate thereof:

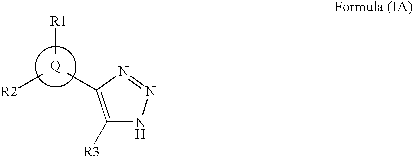

wherein:

Q is a 5- or 6-membered monocyclic ring containing up to two heteroatoms selected from N, O, or S, or an 8- to 11-membered fused bicyclic ring containing up to four heteroatoms selected from N, O, or S;

R¹ and R² are independently selected from H—, Ph-C₀₋₆alkyl-, Het-C₀₋₆alkyl-, C₁₋₆alkyl-, C₁₋₆alkoxy-, C₁₋₆mercaptyl-, Ph-C₀₋₆alkoxy-, Het-C₀₋₆alkoxy-, HO—, R⁴R⁵N-, Het-S—C₀₋₆alkyl-, Ph-S—C₀₋₆alkyl-, HO(CH₂)₁₋₆—, R⁴R⁵N(CH₂)₂₋₆—, R⁴R⁵N(CH₂)₂₋₆O—, R⁶CO₂(CH₂)₀₋₆—, R⁶CO₂(CH₂)₁₋₆O—, R⁶SO₂(CH₂)₁₋₆—, —CF₃, —OCF₃, or halogen, and Ph or Het are substituted with up to five of C₂₋₆alkyl-, C₁₋₆alkoxy-, R⁴R⁵N(CH₂)₁₋₆—, R⁴R⁵N(CH₂)₂₋₆O—, —CO₂R⁶, —CF₃ or, halogen;

R³ is H—, halogen, or R³ and Q together form a bicyclic or tricyclic saturated or unsaturated fused ring system wherein R³ is —C—, or —C═C—; and

R⁴, R⁵, and R⁶ are independently selected from H—, C₂₋₆alkyl-, C₃₋₆alkenyl-, C₃₋₆alkynyl-, Ph-C₀₋₆alkyl-, Het-C₀₋₆alkyl-, or C₃₋₇cycloalkyl-C₀₋₆alkyl-.

In another aspect, the present invention is to a method of inhibiting MetAP2 in the treatment of angiogenesis-mediated diseases, all in mammals, preferably humans, comprising administering to such mammal in need thereof, a compound of formula (IA), or a pharmaceutically active salt thereof.

In yet another aspect, the present invention is to a pharmaceutical composition comprising a compound of formula (I) or formula (IA) and a pharmaceutically acceptable carrier therefor. In particular, the pharmaceutical compositions of the present invention are used for treating MetAP2-mediated diseases.

In a further aspect, the present invention is to novel intermediates useful in the preparation of the compounds of this invention.

### DETAILED DESCRIPTION OF THE INVENTION

It has now been discovered that substituted 1,2,3-triazoles of formula (I) and formula (IA) are inhibitors of MetAP2. It has also now been discovered that selective inhibition of MetAP2 enzyme mechanisms by treatment with the inhibitors of formula (I) and formula (IA), or a pharmaceutically acceptable salt thereof, represents a novel therapeutic and preventative approach to the treatment of a variety of disease states, including, but not limited to, cancer, haemangioma, proliferative retinopathy, rheumatoid arthritis, atherosclerotic neovascularization, psoriasis, ocular neovascularization and obesity.

The term “Ph” represents a phenyl ring. The terms “Het” or “heterocyclic” as used herein interchangeably at all occurrences, mean a stable heterocyclic ring, all of which are either saturated or unsaturated, and consist of carbon atoms and from one to three heteroatoms selected from the group consisting of N, O and S, and wherein the nitrogen may optionally be oxidized or quaternized, and including any bicyclic group in which any of the above-defined heterocyclic rings is fused to a benzene ring. Ph and Het must be substituted with up to five of C₂₋₆alkyl-, C₁₋₆alkoxy-, R⁴R⁵N(CH₂)₁₋₆—, R⁴R⁵N(CH₂)₂₋₆O—, —CO₂R⁶, —CF₃ or, halogen.

The term “C₁₋₆alkyl” as used herein at all occurrences means a substituted and unsubstituted, straight or branched chain radical of 1 to 6 carbon atoms, unless the chain length is limited thereto, including, but not limited to methyl, ethyl, n-propyl, isopropyl, n-butyl, isobutyl and t-butyl, pentyl, n-pentyl, isopentyl, neopentyl and hexyl and the simple aliphatic isomers thereof. Any C₁₋₆alkyl group may be optionally substituted independently by one or more of OR⁴, R⁴, NR⁴R⁵.

The term “C₃₋₇cycloalkyl” as used herein at all occurrences means substituted or unsubstituted cyclic radicals having 3 to 7 carbons, including but not limited to cyclopropyl, cyclopentyl, cyclohexyl and cycloheptyl radicals.

The term “C₂₋₆alkenyl” as used herein at all occurrences means an alkyl group of 2 to 6 carbons wherein a carbon-carbon single bond is replaced by a carbon-carbon double bond. C₂₋₆alkenyl includes ethylene, 1-propene, 2-propene, 1-butene, 2-butene, isobutene and the several isomeric pentenes and hexenes. Both cis and trans isomers are included within the scope of this invention. Any C₂₋₆alkenyl group may be optionally substituted independently by one or more of Ph-C₀₋₆alkyl-, Het-C₀₋₆ alkyl-, C₁₋₆alkyl-, C₁₋₆alkoxy-, C₁₋₆mercaptyl-, Ph-C₀₋₆alkoxy-, Het-C₀₋₆alkoxy-, HO—, R⁴R⁵N—, Het-S—C₀₋₆alkyl-, Ph-S—C₀₋₆alkyl-, HO(CH₂)₁₋₆—, R⁴R⁵N(CH₂)₂₋₆—, R⁴R⁵N(CH₂)₂₋₆O—, R⁶CO₂(CH₂)₀₋₆—, R⁶CO₂(CH₂)₁₋₆O—, R⁶SO₂(CH₂)₁₋₆—, —CF₃, —OCF₃, or halogen.

The term “C₂₋₆alkynyl” as used herein at all occurrences means an alkyl group of 2 to 6 carbons wherein one carbon-carbon single bond is replaced by a carbon-carbon triple bond. C₂₋₆alkynyl includes acetylene, 1-propyne, 2-propyne, 1-butyne, 2-butyne, 3-butyne and the simple isomers of pentyne and hexyne.

The term “alkoxy” is used herein at all occurrences to mean a straight or branched chain radical of 1 to 6 carbon atoms, unless the chain length is limited thereto, bonded to an oxygen atom, including, but not limited to, methoxy, ethoxy, n-propoxy, isopropoxy, and the like.

The term “mercaptyl” is used herein at all occurrences to mean a straight or branched chain radical of 1 to 6 carbon atoms, unless the chain length is limited thereto, bonded to a sulfur atom, including, but not limited to, methylthio, ethylthio, n-propylthio, isopropylthio, and the like.

The terms “hetero” or “heteroatom” as used herein interchangeably at all occurrences mean oxygen, nitrogen and sulfur. The terms “halo” or “halogen” as used herein interchangeably at all occurrences mean F, Cl, Br, and I.

Here and throughout this application the term Co denotes the absence of the substituent group immediately following; for instance, in the moiety PhC₀₋₆alkyl, when C is 0, the substituent is phenyl.

It will be understood that for compounds of formula (I) and formula (IA), the triazole ring can exist in either of two tautomeric forms as shown in FIG. 1. The hydrogen on the triazole ring can exist on either N1 or N3, thus the name for a compound in FIG. 1 can be any of the following: 4-(Q)-1H-1,2,3-triazole, 5-(Q)-1H-1,2,3-triazole, 4-(Q)-3H-1,2,3-triazole, 5-(Q)-3H-1,2,3-triazole. These compounds are equivalent and, for consistency and simplicity, are represented throughout this application as one structure and one name (4-(Q)-1H-1,2,3-triazole).

[image]

The term “Q” is used herein to represent a 5- or 6-membered monocyclic ring optionally containing up to two heteroatoms selected from N, O, or S, or an 8- to 11-membered fused bicyclic ring optionally containing up to four heteroatoms selected from N, O, or S. A bicyclic ring is defined as two rings that are fused together by two adjacent atoms. Suitably, the ring may be saturated or unsaturated, wherein the nitrogen may optionally be oxidized or quaternized. It will be understood that if Q is a heterocyclic ring, it may be attached to the triazole ring through any heteroatom or carbon atom of Q which results in the creation of a stable structure.

Examples of Q include, but are not limited to phenyl, napthyl, piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, pyridinyl, pyrazinyl, oxazolidinyl, oxazolinyl, oxazolyl, isoxazolyl, morpholinyl, thiazolidinyl, thiazolinyl, thiazolyl, quinuclidinyl, indolyl, quinolinyl, isoquinolinyl, benzimidazolyl, benzopyranyl, benzoxazolyl, furyl, pyranyl, tetrahydrofuryl, tetrahydropyranyl, thienyl, benzoxazolyl, benzofuranyl, benzothiophenyl, thiamorpholinyl sulfoxide, thiamorpholinyl sulfone, oxadiazolyl, triazolyl, thiadiazolyl, oxadiazolyl, isoxazolyl, isothiazolyl, imidazolyl, pyridazinyl, pyrimidinyl and triazinyl which moieties are available commercially or can be made by routine chemical synthesis and are stable.

Suitably, Q is a 5- or 6-membered unsaturated ring or a 9-membered bicyclic ring. Preferably, Q is thiophene, phenyl, pyridine, benzofuran, or benzo[1,3]dioxole.

It will be understood that for compounds of formula (I), Q is substituted by up to eight of R¹ and if Q is Ph, Q is additionally substituted by one or more R².

It will be understood that for compounds of this invention, Q is substituted by up to eight substituents, selected independently from R¹ and R².

Suitably, R¹ is H—, Ph-C₀₋₆alkyl-, Het-C₀₋₆ alkyl-, C₁₋₆alkyl-, C₁₋₆alkoxy-, C₁₋₆mercaptyl-, Ph-C₀₋₆alkoxy-, Het-C₀₋₆alkoxy-, HO—, R⁴R⁵N—, Het-S—C₀₋₆alkyl-, Ph-S—C₀₋₆alkyl-, HO(CH₂)₁₋₆—, R⁴R⁵N(CH₂)₂₋₆—, R⁴R⁵N(CH₂)₂₋₆O—, R⁶CO₂(CH₂)₀₋₆-, R⁶CO₂(CH₂)₁₋₆O—, R⁶S0₂(CH₂)₁₋₆—, —CF₃, —OCF₃, or halogen, and Ph or Het are substituted with up to five of C₂₋₆alkyl-, C₁₋₆alkoxy—, R⁴R⁵N(CH₂)₁₋₆—, R⁴R⁵N(CH₂)₂₋₆)—, —CO₂R⁶, —CF₃ or, halogen. Preferably, R¹ is halogen, C₁₋₆alkyl-, C₁₋₆alkoxy-, or —OH. More preferably, R¹ is bromine, chlorine, methyl, ethyl, methoxyl, or hydroxyl.

Suitably, R² is Ph-C₀₋₆alkyl-, Het-C₀₋₆ alkyl-, C₅₋₆alkyl-, C₂₋₆alkoxy-, C₁₋₆mercaptyl-, Ph-C₀₋₆alkoxy-, Het-C₀₋₆alkoxy-, HO—, R⁴R⁵N—, Het-S—C₀₋₆alkyl-, Ph-S—C₀₋₆alkyl-, HO(CH₂)₁₋₆-, R⁴R⁵N(CH₂)₂₋₆—, R⁴R⁵N(CH₂)₂₋₆O—, R⁶CO₂(CH₂)₀₋₆—, R⁶CO₂(CH₂)₁₋₆O—, R⁶SO₂(CH₂)₁₋₆—, —CF₃ or —OCF₃, and Ph or Het are substituted with up to five of C₂₋₆alkyl-, C₁₋₆alkoxy-, R⁴R⁵N(CH₂)₁₋₆—, R⁴R⁵N(CH₂)₂₋₆O—, —CO₂R⁶, —CF₃ or, halogen; wherein R⁴, R⁵, and R⁶ are independently selected from H, C₂₋₆alkyl, C₃₋₆alkenyl, C₃₋₆alkynyl, Ph-C₀₋₆alkyl, Het-C₀₋₆alkyl, or C₃₋₇cycloalkyl-C₀₋₆alkyl. Preferably, R² is —NR⁴R⁵, —CF₃, Ph-S—C₀₋₆alkyl-, Ph-C₀₋₆alkoxy-. More preferably, R² is benzylamine, propylamine, furan-3-ylmethylamine, furan-2-ylmethylamine, —CF₃, Ph-CH₂—O—, (4-Cl)Ph-S—.

For compounds of formula IA, R³ is suitably H—, halogen, or R³ and Q together form a fused bicyclic or tricyclic saturated or unsaturated ring system wherein R³ is —C—, or —C═C—. Preferably, R³ is hydrogen, bromine, or is fused to Q by —C— to form a dihydro-indenotriazole or by —C═C— to form a napthotriazole or an acetonapthotriazole.

Suitably, R⁴, R⁵, and R⁶ are independently selected from H—, C₂₋₆alkyl-, C₃₋₆alkenyl-, C₃₋₆alkynyl-, Ph-C₀₋₆alkyl-, Het-C₀₋₆alkyl-, or C₃₋₇cycloalkyl-C₀₋₆alkyl-. Preferably R⁴, R⁵, and R⁶ are independently selected hydrogen, benzyl, furanyl, and propyl.

Further, it will be understood that when a moiety is “optionally substituted” the moiety may have one or more optional substituents, each optional substituent being independently selected.

Suitably, pharmaceutically acceptable salts of formula (I) include, but are not limited to, salts with inorganic acids such as hydrochloride, sulfate, phosphate, diphosphate, hydrobromide, and nitrate, or salts with an organic acid such as malate, maleate, fumarate, tartrate, succinate, citrate, acetate, lactate, methanesulfonate, p-toluenesulfonate, palmitate, salicylate, and stearate.

The compounds of the present invention may contain one or more asymmetric carbon atoms and may exist in racemic and optically active forms. The stereocenters may be (R), (S) or any combination of R and S configuration, for example, (R,R), (R,S), (S,S) or (S,R). All of these compounds are within the scope of the present invention.

Novel intermediates useful in making compounds of this invention are as follows:

4-ethynyl-benzo[1,3]dioxole;

1-(4-chloro-phenylsulfanyl)-2-ethynylbenzene;

(3-phenyl-propyl)-(3-ethynylphenyl)amine;

phenethyl-(3-ethynylphenyl)-amine;

furan-2-ylmethyl-(3-ethynylphenyl)-amine;

furan-3-ylmethyl-(3-ethynylphenyl)-amine;

napthalene-1-ylmethyl-(3-ethynylphenyl)-amine; and

napthalene-2-ylmethyl-(3-ethynylphenyl)-amine.

The intermediates useful for this invention were made according to the Schemes herein.

Among the preferred compounds of the formula (IA) are the following, compounds:

3-(1H-1,2,3-triazol-4-yl)-phenol;

4-(3-iodophenyl)-1H-1,2,3-triazole;

4-(2-fluorophenyl)-1H-1,2,3-triazole;

4-(4-n-butylphenyl)-1H-1,2,3-triazole;

4-(2-chlorophenyl)-1H-1,2,3-triazole;

N-(3-[1H-1,2,3-triazol-4-yl]phenyl)benzamide;

3-(1H-1,2,3-triazol-4-yl)-phenylamine;

N-(3-[1H-1,2,3-triazol-4-yl]phenyl)acetamide;

4-(4-trifouoromethylphenyl)-1H-1,2,3-triazole;

4-(3-trifouoromethylphenyl)-1H-1,2,3-triazole;

4-(4-n-propylphenyl)-1H-1,2,3-triazole;

4-(4-methoxyphenyl)-1H-1,2,3-triazole;

4-(3-methylphenyl)-1H-1,2,3-triazole;

2-(1H-1,2,3-triazol-4-yl)-pyridine;

4-(4-chlorophenyl)-1H-1,2,3-triazole;

4-(4-ethylphenyl)-1H-1,2,3-triazole;

4-(1H-1,2,3-triazol-4-yl)-phenylamine;

4-(4-methylphenyl)-1H-1,2,3-triazole;

2-(1H-1,2,3-triazol-4-yl)-5-methylpyridine;

2-(1H-1,2,3-triazol-4-yl)-4-methyl-pyridine;

1-(1H-1,2,3-triazol-4-yl)cyclohexanol;

4-(thiophen-2-yl)-1H-1,2,3-triazole;

4-(thiophen-3-yl)-1H-1,2,3-triazole;

4-(2-methylphenyl)-1H-1,2,3-triazole;

4-(1,3-dimethylphenyl)-1H-1,2,3-triazole;

4-(4-bromophenyl)-1H-1,2,3-triazole;

4-(1,3-dichlorophenyl)-1H-1,2,3-triazole;

4-(1-biphenyl-2-yl)-1H-1,2,3-triazole;

4-(2-benzyloxy-phenyl)-1H-1,2,3-triazole;

2-(1H-1,2,3-triazol-4-yl)-6-methylpyridine;

3-(1H-1,2,3-triazol-4-yl)-pyridine;

4-(1H-1,2,3-triazol-4-yl)-pyridine;

4-(2-methoxyphenyl)-1H-1,2,3-triazole;

4-(2-bromophenyl)-1H-1,2,3-triazole;

4-benzo[1,3]dioxol-5-yl-1H-1,2,3-triazole;

2-(1H-1,2,3-triazol-4-yl)-benzofuran;

4-benzo[1,3]dioxol-4-yl-1H-1,2,3-triazole;

4-(2-[4-chloro-phenylsulfanyl]-phenyl)-1H-1,2,3-triazole;

(3-phenyl-propyl)-(3-[1H-1,2,3-triazol-4-yl]phenyl)amine;

phenethyl-(3-[1H-1,2,3-triazol-4-yl]phenyl)amine;

furan-2-ylmethyl-(3-[1H-1,2,3-triazol-4-yl]phenyl)amine;

furan-3-ylmethyl-(3-[1H-1,2,3-triazol-4-yl]phenyl)amine;

napthalene-1-ylmethyl-(3-[1H-1,2,3-triazol-4-yl]phenyl)amine;

napthalene-2-ylmethyl-(3-[1H-1,2,3-triazol-4-yl]phenyl)amine;

4-(1H-1,2,3-triazol-4-yl)-phenol;

benzyl-(3-[1H-1,2,3-triazol-4-yl]phenyl)amine;

4-(4-fluorophenyl)-1H-1,2,3-triazole;

2-bromo-5-(1H-1,2,3-triazol-4-yl)-phenol;

2,6-dibromo-5-(1H-1,2,3-triazol-4-yl)-phenol;

2,4-dibromo-5-(1H-1,2,3-triazol-4-yl)-phenol;

2-(5-bromo-1H-1,2,3-triazol-4-yl)-4-methyl-pyridine;

1H-naptho[1,2-d]-1,2,3-triazole;

2,8-dihydro-indeno[1,2-d]-1,2,3-triazole;

4-phenyl-1H-1,2,3-triazole; and

5,5a,6,8-tetrahydro-4H-acenaphtho[4,5-d]-1,2,3-triazole.

Among the most preferred compounds of the formula (IA) are the following compounds:

4-(3-iodophenyl)-1H-1,2,3-triazole;

4-(2-fluorophenyl)-1H-1,2,3-triazole;

4-(2-chlorophenyl)-1H-1,2,3-triazole;

4(3-methylphenyl)-1H-1,2,3-triazole;

4-4-chlorophenyl)-1H-1,2,3-triazole;

4-(4-ethylphenyl)-1H-1,2,3-triazole;

4-(4-methylphenyl)-1H-1,2,3-triazole;

2-(1H-1,2,3-triazol-4-yl)-5-methylpyridine;

2-(1H-1,2,3-triazol-4-yl)-4-methyl-pyridine;

4-(thiophen-3-yl)-1H-1,2,3-triazole;

4-(4-bromophenyl)-1H-1,2,3-triazole;

4-(1,3-dichlorophenyl)-1H-1,2,3-triazole;

2-(1H-1,2,3-triazol-4-yl)-benzofuran;

furan-2-ylmethyl-(3-[1H-1,2,3-triazol-4-yl]phenyl)amine;

furan-3-ylmethyl-(3-[1H-1,2,3-triazol-4-yl]phenyl)amine;

benzyl-(3-[1H-1,2,3-triazol-4-yl]phenyl)amine;

4(4-fluorophenyl)-1H-1,2,3-triazole;

2-bromo-5-(1H-1,2,3-triazol-4-yl)-phenol;

2,4-dibromo-5-(1H-1,2,3-triazol-4-yl)-phenol; and

2-(5-bromo-1H-1,2,3-triazol-4-yl)-4-methyl-pyridine.

Methods of Preparation

Compounds of the formulae (I) or (IA), were prepared by methods analogous to those described in Scheme 1.

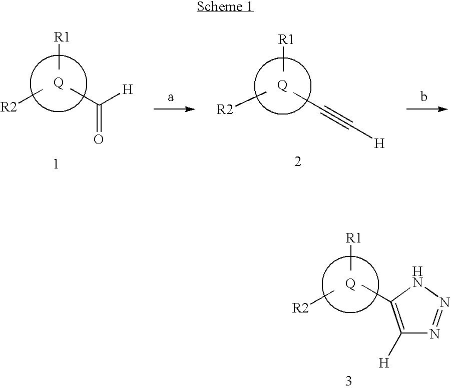

a) P(O)(OMe)₂C(N₂)C(O)CH₃, K₂CO₃, MeOH b) 1. Me₃SiN₃, PhCH₃, 110° C.; 2. H₂O.

An aldehyde (such as 2-thiophenecarboxaldehyde) (1-Scheme1) was treated with 1-diazo-2-oxopropylphosphonate and potassium carbonate in dry methanol to provide 2-Scheme1. Treatment of the acetylene (such as 2-ethynylthiophene) (2-Scheme1) with azidotrimethylsilane in refluxing toluene, followed by addition of water afforded 3-Scheme1.

Compounds of the formulae (I) or (IA), were R² is NHR⁴ were prepared by methods analogous to those described in Scheme 2.

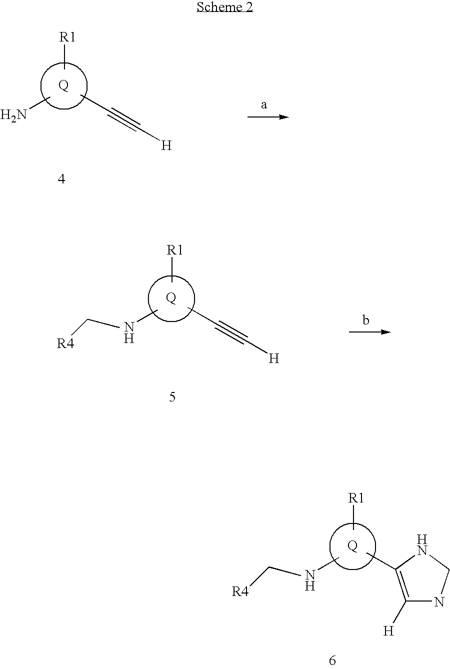

a) R4-C(O)H, Na(OAc)₃BH, ClCH₂CH₂Cl, AcOH b) 1. Me₃SiN₃, PhCH₃, 110° C.; 2. H₂O.

An alkynyl aniline (such as 3-ethynylphenylamine) was substituted by a reductive amination reaction with an aldehyde to provide 5-Scheme2. Treatment of the acetylene (5-Scheme2) with azidotrimethylsilane in refluxing toluene, followed by addition of water afforded 6-Scheme2.

Formulation of Pharmaceutical Compositions

The pharmaceutically effective compounds of this invention (and the pharmaceutically acceptable salts thereof) are administered in conventional dosage forms prepared by combining a compound of this invention of formula (I) or (IA) (“active ingredient”) in an amount sufficient to treat cancer, haemangioma, proliferative retinopathy, rheumatoid arthritis, atherosclerotic neovascularization, psoriasis, ocular neovascularization or obesity (“MetAp2-mediated disease states”) with standard pharmaceutical carriers or diluents according to conventional procedures well known in the art. These procedures may involve mixing, granulating and compressing or dissolving the ingredients as appropriate to the desired preparation.

The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time delay material well known to the art, such as glyceryl monostearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg to about 1000 mg. When a liquid carrier is used, the preparation will be in the form of a syrup, emulsion, soft gelatin capsule, sterile injectable liquid such as an ampule or nonaqueous liquid suspension.

The active ingredient may also be administered topically to a mammal in need of treatment or prophylaxis of MetAP2-mediated disease states. The amount of active ingredient required for therapeutic effect on topical administration will, of course, vary with the compound chosen, the nature and severity of the disease state being treated and the mammal undergoing treatment, and is ultimately at the discretion of the physician. A suitable dose of an active ingredient is 1.5 mg to 500 mg for topical administration, the most preferred dosage being 1 mg to 100 mg, for example 5 to 25 mg administered two or three times daily.

By topical administration is meant non-systemic administration and includes the application of the active ingredient externally to the epidermis, to the buccal cavity and instillation of such a compound into the ear, eye and nose, and where the compound does not significantly enter the blood stream. By systemic administration is meant oral, intravenous, intraperitoneal and intramuscular administration.

While it is possible for an active ingredient to be administered alone as the raw chemical, it is preferable to present it as a pharmaceutical formulation. The active ingredient may comprise, for topical administration, from 0.001% to 10% w/w, e.g. from 1% to 2% by weight of the formulation although it may comprise as much as 10% w/w but preferably not in excess of 5% w/w and more preferably from 0.1% to 1% w/w of the formulation.

The topical formulations of the present invention, both for veterinary and for human medical use, comprise an active ingredient together with one or more acceptable carrier(s) therefor and optionally any other therapeutic ingredient(s). The carrier(s) must be ‘acceptable’ in the sense of being compatible with the other ingredients of the formulation and not deleterious to the recipient thereof.

Formulations suitable for topical administration include liquid or semi-liquid preparations suitable for penetration through the skin to the site of inflammation such as liniments, lotions, creams, ointments or pastes, and drops suitable for administration to the eye, ear or nose.

Drops according to the present invention may comprise sterile aqueous or oily solutions or suspensions and may be prepared by dissolving the active ingredient in a suitable aqueous or alcoholic solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention ate semi-solid formulations of the active ingredient for external application. They may be made by mixing the active ingredient in finely divided or powdered form, alone, or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol. The formulation may incorporate any suitable surface-active agent such as an anionic, cationic or non-ionic surfactant such as esters or polyoxyethylene derivatives thereof. Suspending agents such as natural gums, cellulose derivatives or inorganic materials such as silicaceous silicas, and other ingredients such as lanolin, may also be included.

The active ingredient may also be administered by inhalation. By “inhalation” is meant intranasal and oral inhalation administration. Appropriate dosage forms for such administration, such as an aerosol formulation or a metered dose inhaler, may be prepared by conventional techniques. The daily dosage amount of the active ingredient administered by inhalation is from about 0.1 mg to about 100 mg per day, preferably about 1 mg to about 10 mg per day.

In one aspect, this invention relates to a method of treating cancer, haemangioma, proliferative retinopathy, rheumatoid arthritis, atherosclerotic neovascularization, psoriasis, ocular neovascularization or obesity, all in mammals, preferably humans, which comprises administering to such mammal an effective amount of a MetAP2 inhibitor, in particular, a compound of this invention.

By the term “treating” is meant either prophylactic or therapeutic therapy. Such compound can be administered to such mammal in a conventional dosage form prepared by combining the compound of this invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. The compound is administered to a mammal in need of treatment for cancer, haemangioma, proliferative retinopathy, rheumatoid arthritis, atherosclerotic neovascularization, psoriasis, ocular neovascularization or obesity, in an amount sufficient to decrease symptoms associated with these disease states. The route of administration may be oral or parenteral.

The term parenteral as used herein includes intravenous, intramuscular, subcutaneous, intra-rectal, intravaginal or intraperitoneal administration. The subcutaneous and intramuscular forms of parenteral administration are generally preferred. The daily parenteral dosage regimen will preferably be from about 30 mg to about 300 mg per day of active ingredient. The daily oral dosage regimen will preferably be from about 100 mg to about 2000 mg per day of active ingredient.

It will be recognized by one of skill in the art that the optimal quantity and spacing of individual dosages of a compound of this invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of the compound given per day for a defined number of days, can be ascertained by those skilled in the art using conventional course of treatment determination tests.

### EXAMPLES

The invention will now be described by reference to the following examples which are merely illustrative and are not to be construed as a limitation of the scope of the present invention. In the Examples, proton NMR spectra were performed upon a Bruker 400 MHz NMR spectrometer, unless otherwise indicated.

### Example 1

Preparation of 3-(1H-1,2,3-triazol-4-yl)-phenol

To a stirring solution of 3-ethynylphenol (0.55 g, 4.0 mmol) in 4 ml of toluene under an inert atmosphere was added trimethylsilylazide (1 ml, 8 mmol). The resulting solution was heated to reflux for 3 days. To this mixture was added water (1 ml) and after evaporation, the resulting residue was purified by preparative HPLC to afford the title compound as a white solid (0.12 g, 18%). ¹H-NMR (400 MHz, CD₃OD): δ 8.09 (s, 1H, 7.27 (m, 3H), 6.81 (m, 1H). MS (ESI) 162.2 (M+H)⁺. (This procedure was adapted from Tanaka, Y.; Velen, S. R.; Miller, S. I. Tetrahedron, 1973, 29, 3271.)

### Example 2

Preparation of 4-(3-iodophenyl)-1H-1,2,3-triazole

Following the procedure of Example 1, except substituting 1-ethynyl-3-iodobenzene for 3-ethynylphenol, the title compound was prepared as a white solid (20%). ¹H-NMR (400 MHz, CDCl₃): δ 8.21 (s, 1H), 7.98 (s, 1H), 7.81 (d, J=7.8 Hz, 1H), 7.73 (d, J=8.1 Hz, 1H), 7.21 (t, J=7.8 Hz, 1H). MS (ESI) 272.0 (M+H)⁺.

### Example 3

Preparation of 4-(2-fluorophenyl)-1H-1,2,3-triazole

Following the procedure of Example 1, except substituting 1-ethynyl-2-fluorobenzene for 3-ethynylphenol, the title compound was prepared as a white solid (21%). ¹H-NMR (400 MHz, CDC1₃): δ 11.54 (br s, 1H), 8.19 (s, 1H), 8.11 (t, J=7.5 Hz, 1H), 7.18-7.40 (m, 3H). MS (ESI) 164.2 (M+H)⁺.

### Example 4

Preparation of 4-(4-n-butylphenyl)-1H-1,2,3-triazole

Following the procedure of Example 1, except substituting 1-ethynyl-4-n-butylbenzene for 3-ethynylphenol, the title compound was prepared as a white solid (16%). ¹H-NMR (400 MHz, CD₃OD): δ 8.11 (s, 11H), 7.74 (d, J=7.8 Hz, 2H), 7.28 (d, J=8.0 Hz, 2H), 2.67 (t, J=7.6 Hz, 2H), 1.61-1.69 (m, 2H), 1.37-1.43 (m, 2H), 0.97 (t, J=7.3 Hz, 3H). MS (ESI) 202.2 (M+H)⁺.

### Example 5

Preparation of 4-(2-chlorophenyl)-1H-1,2,3-triazole

Following the procedure of Example 1, except substituting 1-chloro-2-ethynylbenzene for 3-ethynylphenol, the title compound was prepared as a white solid (35%). ¹H-NMR (400 MHz, CD₃OD): δ 8.29 (s, 1H), 7.90 (d, J=7.0 Hz, 1H), 7.53-7.56 (m, 1H), 737-7.44 (m, 2M). MS (ESI) 180.0 (M+H)⁺.

### Example 6

Preparation of N-(3-[1H-1,2,3-triazol-4-yl]phenyl)benzamide

Following the procedure of Example 1, except substituting N-(3-ethynylphenyl)benzamide for 3-ethynylphenol, the title compound was prepared as a white solid (12%). ¹H-NMR (400 MHz, CD₃OD): δ 8.18-8.20 (m, 2H), 7.93-8.00 (m, 2H), 7.45-7.76 (m, 6H). MS (ESI) 265.2 (M+H)⁺.

### Example 7

Preparation of 3-(1H-1,2,3-triazol-4-yl)-phenylamine

Following the procedure of Example 1, except substituting 3-ethynyl-phenylamine for 3-ethynylphenol, the title compound was prepared as a tan solid (19%). ¹H-NMR (400 MHz, CD₃OD): δ 8.05 (s, 1H), 7.12-7.20 (m, 3H), 6.73-6.75 (m, 1H). MS (ESI) 161.2 (M+H)⁺.

### Example 8

Preparation of N-(3-[1H-1,2,3-triazol-4-yl]phenyl)acetamide

Following the procedure of Example 1, except substituting N-(3-ethynylphenyl)acetamide for 3-ethynylphenol, the title compound was prepared as a tan solid (49%). ¹H-NMR (400 MHz, DMSO-d₆): δ10.04 (s, 1H), 8.11-8.50 (m, 21H), 7.35-7.58 (m, 3H), 2.06 (s, 3H). MS (ESI) 203.2 (M+H)⁺.

### Example 9

Preparation of 4-(4-trifouoromethylphenyl)-1H-1,2,3-triazole

Following the procedure of Example 1, except substituting 1-ethynyl-4-trifouoromethylphenyl for 3-ethynylphenol, the title compound was prepared as a white solid (50%). ¹H-NMR (400 MHz, CD₃OD): δ 8.30 (s, 1H), 8.06 (d, J=8.2 Hz, 2H), 7.76 (d, J=8.2 Hz, 2H). MS (ESI) 214.2 (M+H)⁺.

### Example 10

Preparation of 4-(3-trifouoromethylphenyl)-1H-1,2,3-triazole

Following the procedure of Example 1, except substituting 1-ethynyl-3-trifouoromethylphenyl for 3-ethynylphenol, the title compound was prepared as a white solid (16%). ¹H-NMR (400 MHz, CD₃OD): δ 8.32, (s, 1H), 8.10-8.18 (m, 2H), 7.64-7.68 (m, 1H). MS (ESI) 214.2 (M+H)⁺.

### Example 11

Preparation of 4-(4-n-propylphenyl)-1H-1,2,3-triazole Following the procedure of Example 1, except substituting 1-ethynyl-4n-propylbenzene for 3-ethynylphenol, the title compound was prepared as a white solid (26%). ¹H-NMR (400 MHz, CD₃OD): δ 8.11 (s, 1H), 7.74 (d, J=7.5 Hz, 21), 7.28 (d, J=8.0 Hz, 2H), 2.64 (t, J=7.6 Hz, 2H), 1.64-1.73 (m, 2H), 0.97 (t, J=7.3 Hz, 3H). MS (ESI) 188.2 (M+H)⁺.

### Example 12

Preparation of 4-(4-methoxyphenyl)-1H-1,2,3-triazole

Following the procedure of Example 1, except substituting 1-ethynyl-4-methoxybenzene for 3-ethynylphenol, the title compound was prepared as a white solid (34%). 1H-NMR (400 MHz, CDCl₃): δ 7.92 (s, 1H1), 7.76 (d, J=8.8 Hz, 2H), 7.01 (d, J=8.8 Hz, 2H), 3.88 (s, 3H). MS (ESI) 176.2 (M+H)⁺.

### Example 13

Preparation of 4-(3-methylphenyl)-1H-1,2,3-triazole

Following the procedure of Example 1, except substituting 3-ethynyltoluene for 3-ethynylphenol, the title compound was prepared as a white solid (23%). ¹H-NMR (400 MHz, CD₃OD): δ 8.14 (s, 1H), 7.67 (s, 1H), 7.62 (d, J=7.7 Hz, 1), 7.34 (t, J=7.6 Hz, 1H), 7.20 (d, J=7.6 Hz, 1H), 2.41 (s, 3H). MS (ESI) 160.2 (M+H)⁺.

### Example 14

Preparation of 2-(1H-1,2,3-triazol-4-yl)-pyridine

Following the procedure of Example 1, except substituting 2-ethynylpyridine for 3-ethynylphenol, the title compound was prepared as a white solid (16%). ¹H-NMR (400 MHz, CD₃OD): δ 8.60-8.61 (m, 1H), 8.32 (s, 1H), 8.06 (d, J=8.0 Hz, 1H), 7.90-7.95 (m, 1), 7.38-7.41 (m, 1H). MS (ESI) 147.2 (M+H)⁺.

### Example 15

Preparation of 4-(4-chlorophenyl)-1H-1,2,3-triazole

Following the procedure of Example 1, except substituting 1-chloro-4-ethynylbenzene for 3-ethynylphenol, the title compound was prepared as a white solid (35%). ¹H-NMR (400 MHz, CD₃OD): δ 8.18 (s, 1H), 7.85 (d, J=8.6 Hz, 2H), 7.47 (d, J=8.7 Hz, 2H). MS (ESI) 180.0 (M+H)⁺.

### Example 16

Preparation of 4-(4-ethylphenyl)-1H-1,2,3-triazole

Following the procedure of Example 1, except substituting 1-ethyl-4-ethynylbenzene for 3-ethynylphenol, the title compound was prepared as a white solid (11%). ¹H-NMR (400 MHz, CD₃OD): δ 8.11 (s, 1H), 7.74 (d, J=8.2 Hz, 2H), 7.30 (d, J=8.2 Hz, 2H), 2.69 (q, J=7.6, 2H), 1.27 (t, J=7.6 Hz, 3). MS (ESI) 174.2 (M+H)⁺.

### Example 17

Preparation of 4-(1H-1,2,3-triazol-4-yl)-phenylamine

Following the procedure of Example 1, except substituting 4-ethynylphenylamine for 3-ethynylphenol, the title compound was prepared as an orange solid (9%). ¹H-NMR (400 MHz, CD₃OD): δ 7.94 (s, 1H), 7.54 (d, J=8.6 Hz, 2H), 6.78 (d, J=8.6 Hz, 2H). MS (ESI) 161.2 (M+H)⁺.

### Example 18

Preparation of 4-(4-methylphenyl)-1H-1,2,3-triazole

Following the procedure of Example 1, except substituting 4-ethynyltoluene for 3-ethynylphenol, the title compound was prepared as a white solid (14%). ¹H-NMR (400 MHz, CDCl₃): δ 7.96 (s, 1H), 7.73 (d, J=8.0 Hz, 2H), 7.28-7.30 (m, 21), 1.57 (s, 3H). MS (ESI) 160.2 (M+H)⁺.

### Example 19

Preparation of 2-(1H-1,2,3-triazol-4-yl)-5-methylpyridine Following the procedure of Example 1, except substituting 2-ethynyl-5-methylpyridine (Sakamoto, T.; Nagata, H.; Kondo, Y.; Sato, K.; Yamanaka, H. Chem. Pharm. Bull. 1984, 32, 4866) for 3-ethynylphenol, the title compound was prepared as a white solid (28%). ¹H-NMR (400 MHz, CD₃OD): δ 8.45 (s, 1H), 8.27 (s, 1H), 7.95 (d, J=8.1 Hz, 1H), 7.76 (d, J=8.1 Hz, 1H), 2.41 (s, 3H). MS (ESI) 161.2 (M+H)⁺.

### Example 20

Preparation of 2-(1H-1,2,3-triazol-4-yl)-4-methyl-pyridine

Following the procedure of Example 1, except substituting 2-ethynyl-4-methylpyridine (Sakamoto, T.; Nagata, H.; Kondo, Y.; Sato, K.; Yamanaka, H. Chem. Pharm. Bull. 1984, 32, 4866) for 3-ethynylphenol, the title compound was prepared as a white solid (54%). ¹H-NMR (400 MHz, CD₃OD): δ 8.45 (d, J=5.1 Hz, 1H), 8.29 (s, 1H), 7.91 (s, 1H), 7.23 (d, J=5.1 Hz, 1H), 2.46 (s, 3H). MS (ESI) 161.2 (M+H)⁺.

### Example 21

Preparation of 1-(1H-1,2,3-triazol-4-yl)cyclohexanol

Following the procedure of Example 1, except substituting 1-ethynylcyclohexanol for 3-ethynylphenol, the title compound was prepared as a white solid (10%). ¹H-NMR (400 MHz, CD₃OD): δ 7.70 (s, 1H), 1.39-1.99 (m, 10H). MS (ESI) 168.2 (M+H)⁺.

### Example 22

Preparation of 4-(thiophen-2-yl)-1H-1,2,3-triazole

a) 2-ethynylthiophene

To a stirring solution of 2-thiophenecarboxaldehyde (0.33 g, 3.0 mmol) in dry methanol (30 ml) was added potassium carbonate (0.87 g, 6.3 mmol) and 1-diazo-2-oxopropylphosphonate (0.78 g, 4.1 mmol, Calant, P.; D'Haenens, L.; Vandewalle, M. Synth. Commun. 1984, 14, 155). After 4 h of stirring at room temperature, aqueous sodium bicarbonate (5%, 50 ml) and hexanes (50 ml) were added. The organic layer was collected, dried (MgSO₄) and filtered through a short silica plug. Evaporation yielded the title compound as a clear oil. (This procedure was adapted from Muller, S.; Liepold, B.; Roth, G. J.; Bestmann, H. J. Synlett 1996, 521.)

b) 4-(thiophen-2-yl)-1H-1,2,3-triazole

Following the procedure of Example 1, except substituting 2-ethynylthiophene for 3-ethynylphenol, the title compound was prepared as a white solid (2 steps, 7%). ¹H-NMR (400 MHz, CD₃OD): δ 8.05 (s, 1H), 7.43-7.47 (m, 2H), 7.10-7.13 (m, 1H). MS (ESI) 152.2 (M+H)⁺.

### Example 23

Preparation of 4-(thiophen-3-yl)-1H-1,2,3-triazole

Following the procedure of Example 22, except substituting 3-thiophenecarboxaldehyde for 2-thiophenecarboxaldehyde in step a, the title compound was prepared as a white solid (2 steps, 8%). ¹H-NMR (400 MHz, CD₃OD): δ 8.07 (s, 1H), 7.79 (s, 1H), 7.53 (s, 2H). MS (ESI) 152.2 (M+H)⁺.

### Example 24

Preparation of 4-(2-methylphenyl)-1H-1,2,3-triazole

Following the procedure of Example 22, except substituting o-tolualdehyde for 2-thiophenecarboxaldehyde in step a, the title compound was prepared as a white solid (2 steps, 3%). ¹H-NMR (400 MHz, CD₃OD): δ 7.97 (s, 1H), 7.55-7.58 (m, 1H), 7.26-7.33 (m, 3H), 2.44 (s, 3H). MS (ESI) 160.2 (M+H)⁺.

### Example 25

Preparation of 4-(1,3-dimethylphenyl)-1H-1,2,3-triazole

Following the procedure of Example 22, except substituting 2,4-dimethylbenzaldehyde for 2-thiophenecarboxaldehyde in step a, the title compound was prepared as a white solid (2 steps, 3%). ¹H-NMR (400 MHz, CD₃OD): δ 7.92 (s, 1H), 7.44 (d, J=7.8 Hz, 1H), 7.14 (s, M1), 7.10 (d, J=7.3 Hz, 1H), 2.40 (s, 3H), 2.36 (s, 3H). MS (ESI) 174.2 (M+H)⁺.

### Example 26

Preparation of 4-(4-bromophenyl)-1H-1,2,3-triazole

Following the procedure of Example 22, except substituting 4-bromobenzaldehyde for 2-thiophenecarboxaldehyde in step a, the title compound was prepared as a white solid (2 steps, 7%). ¹H-NMR (400 MHz, CD₃OD): δ 8.19 (s, 1H), 7.77 (d, J=8.6 Hz, 2H), 7.61 (d, J=8.6, 2H). MS (ESI) 224.0 (M+H)⁺.

### Example 27

Preparation of 4-(1,3-dichlorophenyl)-1H-1,2,3-triazole

Following the procedure of Example 22, except substituting 2,4-dichlorobenzaldehyde for 2-thiophenecarboxaldehyde in step a, the title compound was prepared as a white solid (2 steps, 6%). ¹H-NMR (400 MHz, CD₃OD): 6 (s, 1H), 7.91-7.94 (m, 1H), 7.61-7.62 (m, 1H) 7.44-7.48 (m, 1H). MS (ESI) 214.0 (M+H)⁺.

### Example 28

Preparation of 4-(1-biphenyl-2-yl)-1H-1,2,3-triazole

Following the procedure of Example 22, except substituting 2-biphenylcarboxaldehyde for 2-thiophenecarboxaldehyde in step a, the title compound was prepared as a clear oil (2 steps, 27%). 1H-NMR (400 MHz, CD₃OD): δ 7.80 (s, 1H), 7.47-7.49 (m, 2H), 7.36-7.40 (m, 4H), 7.20-7.22 (m, 2H), 6.88 (s, 1H). MS (ESI) 222.2 (M+H)⁺.

### Example 29

Preparation of 4-(2-benzyloxy-phenyl)-1H-1,2,3-triazole

Following the procedure of Example 22, except substituting 2-benzyloxybenzaldehyde for 2-thiophenecarboxaldehyde in step a, the title compound was prepared as a white solid (2 steps, 25%). ¹H-NMR (400 MHz, CD₃OD): δ 8.09 (s, 1H), 8.00 (d, J=7.7 Hz, 1H), 7.33-7.43 (m, 6H), 7.20 (d, J=8.3 Hz, if), 7.07 (t, J=7.5, 1H), 5.25 (s, 2H). MS (ESI) 252.2 (M+H)⁺.

### Example 30

Preparation of 2-(1H-1,2,3-triazol-4-yl)-6-methylpyridine

Following the procedure of Example 22, except substituting 6-methyl-2-pyridine carboxaldehyde for 2-thiophenecarboxaldehyde in step a, the title compound was prepared as a clear oil (2 steps, 39%). ¹H-NMR (400 MHz, CD₃OD): δ 8.33 (s, 1H), 7.77-7.85 (m, 2H), 7.26 (d, J=7.4 Hz, 1H), 2.60 (s, 3H). MS (ESI) 161.2 (M+H)⁺.

### Example 31

Preparation of 3-(1H-1,2,3-triazol-4-yl)-pyridine

Following the procedure of Example 22, except substituting 3-pyridine carboxaldehyde for 2-thiophenecarboxaldehyde in step a, the title compound was prepared as a white solid (2 steps, 25%). %). ¹H-NMR (400 MHz, CD₃OD): δ9.06 (s, 1H), 8.54 (d, J=3.4 Hz, 1H), 8.31-8.33 (m, 2H), 7.54 (m, 1H). MS (ESI) 147.2 (M+H)⁺.

### Example 32

Preparation of 4-(1H-1,2,3-triazol-4-yl)-pyridine

Following the procedure of Example 22, except substituting 4-pyridine carboxaldehyde for 2-thiophenecarboxaldehyde in step a, the title compound was prepared as a white solid (2 steps, 15%). ¹H-NMR (400 MHz, CD₃OD): δ 8.61-8.62 (m, 2H), 8.42 (s, 1H), 7.91-7.93 (m, 2H). MS (ESI) 147.2 (M+H)⁺.

### Example 33

Preparation of 4-(2-methoxyphenyl)-1H-1,2,3-triazole

Following the procedure of Example 22, except substituting o-anisaldehyde for 2-thiophenecarboxaldehyde in step a, the title compound was prepared as a white solid (2 steps, 6%). ¹H-NMR (400 MHz, CD₃OD): δ 8.19 (s, 1H), 7.95 (d, J=6.8 Hz, 1H), 7.35-7.40 (m, 1H), 7.14 (d, J=8.3 z, 1H), 7.04-7.08 (m, 1H), 4.90 (s, 3H). MS (ESI) 176.2 (M+H)⁺.

### Example 34

Preparation of 4-(2-bromophenyl)-1H-1,2,3-triazole

Following the procedure of Example 22, except substituting 2-bromobenzaldehyde for 2-thiophenecarboxaldehyde in step a, the title compound was prepared as a white solid (2 steps, 15%). ¹H-NMR (400 MHz, CD₃OD): δ 8.27 (s, 1H), 7.73-7.79 (m, 2H), 7.45-7.79 (m, 1H), 7.30-7.34 (m, 1H).MS (ESI) 224.0 (M+H)⁺.

### Example 35

Preparation of 4-benzo[1,3]dioxol-5-yl-1H-1,2,3-triazole

Following the procedure of Example 22, except substituting piperonal for 2-thiophenecarboxaldehyde in step a, the title compound was prepared as a white solid (2 steps, 10%). ¹H-NMR (400 MHz, CD₃OD): δ 8.05 (s, 1H), 7.32-7.34 (m, 2H), 6.89-6.91 (m, 1M), 6.00 (s, 21). MS (ESI) 190.2 (M+H)⁺.

### Example 36

Preparation of 2-(1H-1,2,3-triazol-4-yl)-benzofuran

Following the procedure of Example 22, except substituting benzofuran-2-carboxaldehyde for 2-thiophenecarboxaldehyde in step a, the title compound was prepared as a white solid (2 steps, 25%). ¹H-NMR (400 MHz, CD₃OD): δ 8.25 (s, 11), 7.65 (d, J=7.6 Hz, 1H), 7.56 (d, J=8.0 Hz, 1H), 7.23-7.36 (m, 3H). MS (ESI) 186.0 (M+H)⁺.

### Example 37

Preparation of 4-benzo[1,3]dioxol-4-yl-1H-1,2,3-triazole

a) 4-ethynyl-benzo[1,3]dioxole

Following the procedure of Example 22, except substituting benzo[1,3]dioxole-4-carbaldehyde for 2-thiophenecarboxaldehyde in step a, the title compound was obtained as an oil (98%). ¹H-NMR (400 MHz, CDC1₃): δ 6.94-6.96 (m, 1H), 6.80-6.85 (m, 2H), 6.05 (s, 2H), 3.30 (s, 1H).

b) 4-benzo[1,3]dioxol-4-yl-1H-1,2,3-triazole

Following the procedure of Example 1, except substituting 4-ethynyl-benzo[1,3]dioxole for 3-ethynylphenol, the title compound was prepared as a white solid (24%). ¹H-NMR (400 MHz, CD₃OD): δ 8.13 (s, 1H), 7.45 (d, J=8.0 Hz, 1H), 6.94-6.97 (m, 1H1), 6.81-6.83 (m, 1H), 6.08 (s, 2H). MS (ESI) 190.2 (M+H)⁺.

### Example 38

Preparation of 4-(2-[4-chloro-phenylsulfanyl]-phenyl)-1H-1,2,3-triazole

a) 1-(4-chloro-phenylsulfanyl)-2-ethynylbenzene

Following the procedure of Example 22, except substituting 2-(4-chlorophenylthio)benzaldehyde for 2-thiophenecarboxaldehyde in step a, the title compound was obtained as an oil (91%). ¹H-NMR (400 MHz, CDCl₃): δ 7.53-7.55 (m, 1H), 7.17-7.40 (m, 6H), 7.03-7.05 (m, 1H), 3.43 (s, 1H).

b) 4-(2-[4-chloro-phenylsulfanyl]-phenyl)-1H-1,2,3-triazole

Following the procedure of Example 1, except substituting 1-(4-chloro-phenylsulfanyl)-2-ethynylbenzene for 3-ethynylphenol, the title compound was prepared as a white solid (21%). 1H-NMR (400 MHz, CD₃OD): δ 8.10 (s, 1H), 7.77-7.79 (m, 1H), 7.39-7.46 (m, 3H), 7.28-7.30 (m, 2H), 7.15-7.17 (m, 2H).MS (ESI) 288.2 (M+H)⁺.

### Example 39

Preparation of (3-phenyl-propyl)-(3-[1H-1,2,3-triazol-4-yl]phenyl)amine

a) (3-phenyl-propyl)-(3-ethynylphenyl)amine

To a stirring solution of 3-ethynylphenylamine (0.59 g, 5.0 mmol) and 3-phenylpropionaldehyde (0.66 g, 5.0 mmol) in 1,2-dichloroethane (15 ml) was added acetic acid (0.29 ml, 5.0 mmol) and sodium triacetoxyborohydride (1.6 g, 7.5 mmol). After stirring at room temperature for 72 h, aqueous sodium bicarbonate (saturated) and diethyl ether were added. The organic layer was washed with additional sodium bicarbonate, dried (NgSO₄) and evaporated. Purification via silica gel chromatography gave the title compound as a clear oil (42%). MS (ESI) 236.2 (M+H)⁺.

b) (3-phenyl-propyl)-(3-[1H-1,2,3-triazol-4-yl]phenyl)amine

Following the procedure of Example 1, except substituting (3-phenyl-propyl)-(3-ethynylphenyl)amine for 3-ethynylphenol, the title compound was prepared as a clear oil (16%). ¹H-NMR (400 MHz, CD₃OD): δ 8.03 (s, 1H), 7.6.62-7.30 (m, 9H), 3.17 (t, J=7.0 Hz, 2H), 2.77 (t, J=7.4 Hz, 2H), 1.97 (t, J=7.7 Hz, 2H). MS (ESI) 279.4 (M+H)⁺.

### Example 40

Preparation of phenethyl-(3-[1H-1,2,3-triazol-4-yl]phenyl)amine

a) phenethyl-(3-ethynylphenyl)-amine

Following the procedure of Example 39, except substituting phenylacetaldehyde for 3-phenylpropionaldehyde in step a, the title compound was prepared as a clear oil (47%). MS (ESI) 222.2 (M+H)⁺.

b) phenethyl-(3-[1H-1,2,3-triazol-4-yl]phenyl)amine

Following the procedure of Example 1, except substituting phenethyl-(3-ethynylphenyl)-amine for 3-ethynylphenol, the title compound was prepared as a clear oil (19%). ¹H-NMR (400 MHz, CD₃OD): δ 8.07 (s, 1H), 7.06-7.31 (m, 8H), 6.67 (d, J=8.1 Hz, 1H), 3.41 (t, J=7.2 Hz, 2H1), 2.94 (t, J=7.1 Hz, 2H). MS (ESI) 265.2 (M+H)⁺.

### Example 41

Preparation of furan-2-ylmethyl-(3-[1H-1,2,3-triazol-4-yl]phenyl)amine

a) furan-2-ylmethyl-(3-ethynylphenyl)-amine

Following the procedure of Example 39, except substituting furfural for 3-phenylpropionaldehyde in step a, the title compound was prepared as a clear oil (75%). MS (ESI) 198.2 (M+H)⁺.

b) furan-2-ylmethyl-(3-[1H-1,2,3-triazol-4-yl]phenyl)amine

Following the procedure of Example 1, except substituting furan-2-ylmethyl-(3-ethynylphenyl)-amine for 3-ethynylphenol, the title compound was prepared as a white solid (18%). ¹H-NMR (400 MHz, CD₃OD): δ 8.06 (s, 1H), 7.43 (d, J=1.0 Hz, 1H), 7.09-7.22 (m, 3H), 6.72 (d, J=8.1 Hz, 1H), 6.34-6.35 (m, 1H), 6.28 (d, J=3.2 Hz, 1H), 4.36 (s, 2H). MS (ESI) 241.2 (M+H)⁺.

### Example 42

Preparation of furan-3-ylmethyl-(3-[1H-1,2,3-triazol-4-yl]phenyl)amine

a) furan-3-ylmethyl-(3-ethynylphenyl)-amine

Following the procedure of Example 39, except substituting 3-furaldehyde for 3-phenylpropionaldehyde in step a, the title compound was prepared as a clear oil (70%). MS (ESI) 198.2 (M+H)⁺.

b) furan-3-ylmethyl-(3-[1H-1,2,3-triazol-4-yl]phenyl)amine

Following the procedure of Example 1, except substituting furan-3-ylmethyl-(3-ethynylphenyl)-amine for 3-ethynylphenol, the title compound was prepared as a white solid (20%). ¹H-NMR (400 MHz, CD₃OD): δ 8.06 (s, 1H), 7.49 (s, 1H), 7.45-7.46 (m, 1H), 7.08-7.22 (m, 3H), 6.71 (dd, J=6.5, 1.5 Hz, 1H), 6.47 (s, 1H) 4.22 (s, 2H). MS. (ESI) 241.2 (M+H)⁺.

### Example 43

Preparation of napthalene-1-ylmethyl-(3-[1H-1,2,3-triazol-4-yl]phenyl)amine

a) napthalene-1-ylmethyl-(3-ethynylphenyl)-amine

Following the procedure of Example 39, except substituting 1-napthaldehyde for 3-phenylpropionaldehyde in step a, the title compound was prepared as a clear oil (80%). MS (ESI) 258.2 (M+H)⁺.

b) napthalene-1-ylmethyl-(3-[1H-1,2,3-triazol-4-yl]phenyl)amine

Following the procedure of Example 1, except substituting napthalene-1-ylmethyl-(3-ethynylphenyl)-amine for 3-ethynylphenol, the title compound was prepared as a white solid (18%). ¹H-NMR (400 MHz, CD₃OD): δ 8.16 (d, J=8.2 Hz, 1H), 7.99 (br s, 1H), 7.91 (d, J=8.1 Hz, 1H), 7.81 (d, J=8.4 Hz, 1H), 7.42-7.60 (m, 4H), 7.09-7.21 (m, 3H) 6.71 (d, J=8.0 Hz, 1H), 4.83 (s, 2H). MS ESI) 301.2 (M+H)⁺.

### Example 44

Preparation of napthalene-2-ylmethyl-(3-[1H-1,2,3-triazol-4-yl]phenyl)amine

a) napthalene-2-ylmethyl-(3-ethynylphenyl)-amine

Following the procedure of Example 39, except substituting 2-napthaldehyde for 3-phenylpropionaldehyde in step a, the title compound was prepared as a clear oil (90%). MS (ESI) 258.2 (M+H)⁺.

b) napthalene-2-ylmethyl-(3-[1H-1,2,3-triazol-4-yl]phenyl)amine

Following the procedure of Example 1, except substituting napthalene-2-ylmethyl-(3-ethynylphenyl)-amine for 3-ethynylphenol, the title compound was prepared as a white solid (15%). ¹H-NMR (400 MHz, CD₃OD): δ 8.01 (s, 1H), 7.80-7.87 (m, 41), 7.41-7.56 (m, 3H), 7.05-7.19 (m, 3H), 6.70 (d, 3=1.6 Hz, 1H), 4.56 (s, 2H). MS (ESI) 301.2 (M+H)⁺.

### Example 45

Preparation of 4-(1H-1,2,3-triazol-4-yl)-phenol

To 4-(4-methoxyphenyl)-1H-1,2,3-triazole (83 mg, 0.5 mmol, from Example 12) was added hydrobromic acid (48% in water, 2 ml) and the solution was heated to 100° C. After three hours, water (10 ml) and ethyl acetate (10 ml) were added. The water layer was washed with ethyl acetate three times and the collected organic layers were dried, filtered, and evaporated. The resulting residue was purified by preparative HPLC to afford the title compound as a white solid (40%). ¹H-NMR (400 MHz, CD₃OD): δ 8.01 (s, 1H), 7.65 (d, J=8.7 Hz, 2H), 6.87 (d, J=8.7 Hz, 2H). MS (ESI) 162.2 (M+H)⁺.

### Example 46

Preparation of benzyl-(3-[1H-1,2,3-triazol-4-yl]phenyl)amine

To a cooled (0° C.) solution of N-(3-[1H-1,2,3-triazol-4-yl]phenyl)benzamide (50 mg, 0.19 mmol, from Example 6) in THF (0.5 ml) and dioxane (0.5 ml) was added lithium aluminum hydride (1.0 M in THF, 0.2 ml) and the reaction was allowed to warm to room temperature overnight. Additional dioxane (1 ml) and lithium aluminum hydride (0.2 ml) were added with heating to 50° C. to force the reaction to completion. Water and Na₂SO₄ were added and the residue was filtered. The filtrate was evaporated and purified by preparative HPLC to afford the title compound as a tan oil (60%). ¹H-NMR (400M, CD₃OD): δ 7.96 (s, 1H), 7.40-7.43 (m, 2H) 7.30-7.35 (m, 2H), 7.04-7.25 (m, 4H), 6.63 (d, J=8.0 Hz, 1H), 4.38 (s, 2H). MS (ESI) 251.2 (M+H)⁺.

### Example 47

Preparation of 4-(4-fluorophenyl)-1H-1,2,3-triazole

a) 1-chloroethynyl-4-fluorobenzene

To a stirring solution of 1-ethynyl-4-fluorobenzene (1.30 g, 10 mmol) in carbon tetrachloride (5 ml) was added potassium carbonate (1.56 g, 11 mmol) and TBAF (0.23 g, 1.0 mmol). After stirring the reaction at RT for 1 h, water (20 ml) was added and the organic material was collected by extraction into chloroform. The combined chloroform extracts were dried (MgSO₄) and evaporated. Purification by silica gel chromatography (100% hexanes) gave the title compound as a clear oil (60%). (This procedure was adapted from Sasson, Y.; Webster, O. W. J. Chem. Soc., Chem. Commun. 1992, 1200.)

b) 4-fluorophenylethynyltriphenylphosphonium chloride

To triphenylphosphine (1.7 g, 6.3 mmol) in ether (50 ml) was added 1-chloroethynyl-4-fluorobenzene (1.0 g, 6.3 mmol). After sitting for 10 days at RT, the white phosphonium salt was collected by filtration (18%). (This procedure was adapted from Tanaka, Y.; Miller, S. I. J. Org. Chem. 1973, 38, 2708.)

c) 4-(4-fluorophenyl)-1H-1,2,3-triazole

To a warm (60° C.) solution of sodium azide (74 mg, 1.1 mmol) in DMF (4 ml) was added 4-fluorophenylethynyltriphenylphosphonium chloride (476 mg, 1.1 mmol) in DMF (4 ml) dropwise. After the mixture was stirred for 3 h at 60° C., the DMF was removed by evaporation. The residue was dissolved in chloroform, filtered, and the filtrate was evaporated to give a yellow solid. This solid was dissolved in ethanol (5.5 ml) and a sodium hydroxide solution (0.25 M, 11 ml) was added. After stirring and heating to 90° C. for 2 h, water (20 ml) was added and the aqueous layer was extracted with chloroform (10 ml×2). (The organic layers were discarded.) The aqueous layer was neutralized with HCl (6 N) and again extracted with chloroform (10 ml×3). The organic layers were combined, dried (MgSO₄) and evaporated. Purification by preparative HPLC to afforded the title compound as a yellow solid (20%). ¹H-NMR (400 MHz, CD₃OD): δ 8.13 (s, 1H), 7.84-7.88 (m, 2H), 7.16-7.21 (m, 2H). MS (ESI) 164.2 (M+H1)⁺. (This procedure was adapted from Tanaka, Y.; Miller, S. I. J. Org. Chem. 1973, 38, 2708.)

### Example 48

Preparation of 2-bromo-5-(1H-1,2,3-triazol-4-yl)-phenol, 2,6-dibromo-5-(1H-1,2,3-triazol-4-yl)-phenol, and 2,4-dibromo-5-(1H-1,2,3-triazol-4-yl)-phenol

To 3-(1H-1,2,3-triazol-4-yl)-phenol (54 mg, 0.33 mmol, from Example 1) in acetic acid (1 ml) was added bromine (18 uL, 0.33 mmol). After 1 h of stirring at RT, water (10 ml) and ethyl acetate (10 ml) were added. The aqueous layer was neutralized with saturated NaHCO₃. The water layer was washed with ethyl acetate three times and the collected organic layers were dried, filtered, and evaporated. The resulting residue was purified by preparative HPLC to afford the three compounds, each as a white solid. 2-bromo-5-(1H-1,2,3-triazol-4-yl)-phenol (14%): ¹H-NMR (400 MHz, CD₃OD): δ 8.12 (s, 1H), 7.53 (d, J=8.2 Hz, 1H), 7.40 (d, J=2.0 Hz, 1H), 7.22 (dd, J=8.2, 2.0 Hz, 1H). MS (ESI) 240.0 (M+H)⁺. 2,6-dibromo-5-(1H-1,2,3-triazol-4-yl)-phenol (7%): ¹H-NMR (400 MHz, CD₃OD): δ 8.24 (s, 1H), 7.56 (d, J=8.3 Hz, 1H), 7.16 (d, J=8.3 Hz, 1H). MS (ESI) 319.9 (M+H)⁺. 2,4-dibromo-5-(1H-1,2,3-triazol-4-yl)-phenol (8%): ¹H-NMR (400 MHz, CD₃OD): δ 8.29 (s, 1H), 7.80 (s, 1H), 7.36 (s, 1H). MS (ESI) 319.9 (M+H)⁺.

### Example 49

Preparation of 2-(5-bromo-1H-1,2,3-triazol-4-yl)-4-methyl-pyridine

Following the procedure of Example 48, except substituting 2-(1H-1,2,3-triazol-4-yl)-4-methyl-pyridine (Example 21) for 3-(1H-1,2,3-triazol-4-yl)-phenol, the title compound was prepared as an orange solid (16%). ¹H-NMR (400 MHz, CD₃OD): δ 8.53 (d, J=5.0 Hz, 1H), 7.92 (s, 1H), 7.32 (d, J=5.0 Hz, 1H), 2.48 (s, 3H). MS (ESI) 239.0 (M+H)⁺.

### Example 50

Preparation of 1H-naptho[1,2-d]-1,2,3-triazole

Morgan, G.; J. Chem. Soc. 1910, 97, 1719. MS (ESI) 170.0 (M+H)⁺.

### Example 51

Preparation of 2,8-dihydro-indeno[1,2-d]-1,2,3-triazole

Rapoport, H.; Chen, H. H. J. Org. Chem. 1960, 25; 313. MS ESI) 158.0 (M+H)⁺.

### Example 52

Preparation of 4-phenyl-1H-1,2,3-triazole

Tanaka, Y.; Velen, S. R.; Miller, S. I. Tetrahedron, 1973, 29, 3271. MS (ESI) 146.0 (M+H)⁺.

### Example 53

Preparation of 5,5a,6,8-tetrahydro-4H-acenaphtho[4,5-d]-1,2,3-triazole Rapoport, H.; Nilsson, W. J. Am. Chem. Soc. 1961; 83,4262. MS OEST) 198.0 (M+H)⁺.

Biological Data:

Direct Spectrophotometric Assays of hMetAP2:

The hMetAP2 activity can be measured by direct spectrophotometric assay methods using alternative substrates, L-methionine-p-nitroanilide (Met-pNA) and L-methionine-7-amido-4-methylcoumarin (Met-AMC). The formation of p-nitroaniline (pNA) or 7-amido-4-methylcoumarin (AMC) was continuously monitored by increasing absorbance or fluorescence at 405 nm and 460 nm, respectively, on a corresponding plate reader. All assays were carried out at 30° C. The fluorescence or spectrophotometric plate reader was calibrated using authentic pNA and AMC from Sigma, respectively. For a typical 96-well plate assay, the increase in the absorbance (at 405 nm for pNA) or the fluorescence emission (λₑₓ=360 nm, λₑₘ=460 nm, for AMC) of a 50 μL assay solution in each well was used to calculate the initial velocity of hMetAP2. Each 50 μL assay solution, contained 50 mM Hepes.Na⁺ (pH 7.5), 100 mM NaCl, 10-100 nM purified hMetAP2 enzyme, and varying amounts of Met-AMC (in 3% DMSO aqueous solution) or Met-pNA. Assays were initiated with the addition of substrate and the initial rates were corrected for the background rate determined in the absence of hMetAP2.

Coupled Spectrophotometric Assays of hMetAP2:

The methionine aminopeptidase activity of hMetAP2 can also be measured spectrophotometrically by monitoring the free L-amino acid formation. The release of N-terminal methionine from a tripeptide (Met-Ala-Ser, Sigma) or a tetrapeptide (et-Gly-Met-Met, Sigma) substrate was assayed using the L-amino acid oxidase (AAO)/horse radish peroxidase (HRP) couple (eq. 1-3a,b). The formation of hydrogen peroxide (H₂O₂) was continuously monitored at 450 nm (absorbance increase of o-Dianisidine (Sigma) upon oxidation, Δε=15,300 M⁻¹cm⁻¹)² and 30° C. in a 96- or 384-well plate reader by a method adapted from Tsunasawa, S. et al.(1997) (eq. 3a). Alternatively, formation of H₂O₂ was followed by monitoring the fluorescence emission increase at 587 nm (Δε=54,000 M⁻¹cm⁻¹, λₑₓ=563 nm, slit width for both excitation and emission was 1.25 mm) and 30° C. using Amplex Red (Molecular Probes, Inc) (Zhou, M. et al. (1997) Anal. Biochem. 253, 162) (eq. 3b). In a total volume of 50 μL, a typical assay contained 50 mM Hepes.Na⁺, pH 7.5, 100 mM NaCl, 10 μM CoCl₂, 1 mM o-Dianisidine or 50 μM Amplex Red, 0.5 units of HRP (Sigma), 0.035 unit of AAO (Sigma), 1 nM hMetAP2, and varying amounts of peptide substrates. Assays were initiated by the addition of hMetAP2 enzyme, and the rates were corrected for the background rate determined in the absence of hMetAP2.

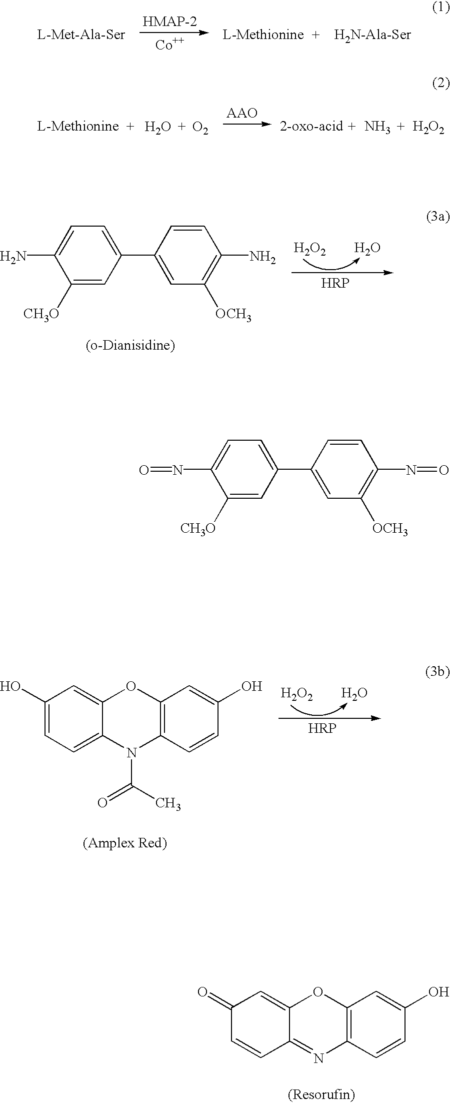

Kinetic Data Analysis:

Data were fitted to the appropriate rate equations using Grafit computer software. Initial velocity data conforming to Michaelis-Menton kinetics were fitted to eq. 4. Inhibition patterns conforming to apparent competitive and non-competitive inhibition were fitted to eq. 5 and eq. 6, respectively.

v=VA/(Kₐ+A) (4)

v=VA/[Kₐ(1+I/Kᵢₛ)+A] (5)

v=VA/[Kₐ(1+I/Kᵢₛ)+A(1+I/Kᵢᵢ)] (6)

In eqs. 4-6, v is the initial velocity, V is the maximum velocity, Kₐ is the apparent Michaelis constant, I is the inhibitor concentration, and A is the concentration of variable substrates. The nomenclature used in the rate equations for inhibition constants is that of Cleland (1963), in which Kᵢₛ and Kᵢᵢ represent the apparent slope and intercept inhibition constants, respectively.

Cell Growth Inhibition Assays:

The ability of MetAP2 inhibitors to inhibit cell growth was assessed by the standard XTT microtitre assay. XTT, a dye sensitive to the pH change of mitochondria in eukaryotic cells, is used to quantify the viability of cells in the presence of chemical compounds. Cells seeded at a given number undergo approximately two divisions on average in the 72 hours of incubation. In the absence of any compound, this population of cells is in exponential growth at the end of the incubation period; the mitochondrial activity of these cells is reflected in the spectrophotometric readout (A450). Viability of a similar cell population in the presence of a given concentration of compound is assessed by comparing the A₄₅₀ reading from the test well with that of the control well. Flat-bottomed 96-well plates are seeded with appropriate numbers of cells (4-6×10³ cells/well in a volume of 200 ul) from trypsinized exponentially growing cultures. In the case of HUVECs, the wells are coated with matrigel prior to establishing the cultures. To “blank” wells is added growth medium only. Cells are incubated overnight to permit attachment. Next day, medium from wells that contain cells is replaced with 180 ul of fresh medium. Appropriate dilutions of test compounds are added to the wells, final DMSO concentration in all wells being 0.2%. Cells plus compound are incubated for an additional 72 hr at 37° C. under the normal growth conditions of the cell line used. Cells are then assayed for viability using standard XTT/PMS (prepared immediately before use: 8 mg XTT (Sigma X-4251) per plate is dissolved in 100 ul DMSO. 3.9 ml H₂O is added to dissolve XTT and 20 ul of PMS stock solution (30 mg/ml) is added from frozen aliquoted stock solution (10 mg of PMS (phenazine methosulfate, Sigma P-9625) in 3.3 ml PBS without cations. These stocks are frozen at −20° C. until use). 50 ul of XTT/PMS solution is added to each well and plates incubated for 90 minutes (time required may vary according to cell line, etc.) at 37° C. until A₄₅₀ is >1.0. Absorbance at 450 nM is determined using a 96-well UV plate reader. Percent viability of cells in each well is calculated from these data (having been corrected for background absorbance). IC₅₀ is that concentration of compound that reduces cell viability to 50% control (untreated) viability.

The compounds of this invention show MetAP2 inhibitor activity having IC₅₀ values in the range of 0.0001 to 100 uM. The full structure/activity relationship has not yet been established for the compounds of this invention. However, given the disclosure herein, one of ordinary skill in the art can utilize the present assays in order to determine which compounds of this invention are inhibitors of MetAP2 and which bind thereto with an IC₅₀ value in the range of 0.0001 to 100 uM.

All publications, including, but not limited to, patents and patent applications cited in this specification, are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

What is claimed is:

1. A method of inhibiting MetAP2 in mammals, comprising administering to a mammal in need of such inhibition, an effective amount of a compound of formula (IA) or a pharmaceutically acceptable salt or solvate thereof: 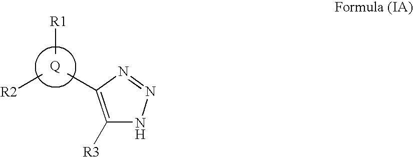
wherein:
Q is a 5- or 6-membered monocyclic ring containing up to two heteroatoms selected from N, O, or S, or an 8- to 11-membered fused bicyclic ring containing up to four heteroatoms selected from N, O, or S;
R¹ and R² are independently selected from H—, Ph-C₀₋₆alkyl-, Het-C₀₋₆alkyl-, C₁₋₆alkyl-, C₁₋₆alkoxy-, C₁₋₆mercaptyl-, Ph-C₀₋₆alkoxy-, Het-C₀₋₆alkoxy-, HO—, R⁴R⁵N-, Het-S—C₀₋₆alkyl-, Ph-S—C₀₋₆alkyl-, HO(CH₂)₁₋₆—, R⁴R⁵N(CH₂)₂₋₆—, R⁴R⁵N(CH₂)₂₋₆O—, R⁶CO₂(CH₂)₀₋₆—, R⁶CO₂(CH₂)₂₋₆O—, R⁶SO₂(CH₂)₁₋₆—, —CF₃, —OCF₃, or halogen, and Ph or Het are substituted with up to five of C₂₋₆alkyl-, C₁₋₆alkoxy-, R⁴R⁵N(CH₂)₁₋₆—, R⁴R⁵N(CH₂)₂₋₆O—, —CO₂R⁶, —CF₃ or, halogen;
R³ is H—, halogen, or R³ and Q together form a fused bicyclic or tricyclic saturated or unsaturated fused ring system wherein R³ is —C—, or —C═C—; and
R⁴, R⁵, and R⁶ are independently selected from H—, C₂₋₆alkyl-, C₃₋₆alkenyl-, C₃₋₆alkynyl-, Ph-C₀₋₆alkyl-, Het-C₀₋₆alkyl-, or C₃₋₇cycloalkyl-C₀₋₆alkyl-.

2. The method of claim 1, wherein the compound of formula (IA) is selected from:
3-(1H-1,2,3-triazol-4-yl)-phenol;
4-(4-n-butylphenyl)-1H-1,2,3-triazole;
N-(3-[1H-1,2,3-triazol-4-yl]phenyl)benzamide;
3-(1H-1,2,3-triazol-4-yl)-phenylamine;
N-(3-[1H-1,2,3-triazol-4-yl]phenyl)acetamide;
4-(4-trifouoromethylphenyl)-1H-1,2,3-triazole;
4-(3-trifouoromethylphenyl)-1H-1,2,3-triazole;
4-(4-n-propylphenyl)-1H-1,2,3-triazole;
4-(4-methoxyphenyl)-1H-1,2,3-triazole;
2-(1H-1,2,3-triazol-4-yl)-pyridine;
4-(1H-1,2,3-triazol-4-yl)-phenylamine;
1-(1H-1,2,3-triazol-4-yl)cyclohexanol;
4-(thiophen-2-yl)-1H-1,2,3-triazole;
4-(2-methylphenyl)-1H-1,2,3-triazole;
4-(1,3-dimethylphenyl)-1H-1,2,3-triazole;
4-(1-biphenyl-2-yl)-1H-1,2,3-triazole;
4-(2-benzyloxy-phenyl)-1H-1,2,3-triazole;
2-(1H-1,2,3-triazol-4-yl)-6-methylpyridine;
3-(1H-1,2,3-triazol-4-yl)-pyridine;
4-(1H-1,2,3-triazol-4-yl)-pyridine;
4-(2-methoxyphenyl)-1H-1,2,3-triazole;
4-(2-bromophenyl)-1H-1,2,3-triazole;
4-benzo[1,3]dioxol-5-yl-1H-1,2,3-triazole;
4-benzo[1,3]dioxol-4-yl-1H-1,2,3-triazole;
4-(2-[4-chloro-phenylsulfanyl]-phenyl)-1H-1,2,3-triazole;
(3-phenyl-propyl)-(3-[1H-1,2,3-triazol-4-yl]phenyl)amine; phenethyl-(3-[1H-1,2,3-triazol-4-yl]phenyl)amine;
napthalene-1-ylmethyl-(3-[1H-1,2,3-triazol-4-yl]phenyl)amine;
napthalene-2-ylmethyl-(3-[1H-1,2,3-triazol-4-yl]phenyl)amine;
4-(1H-1,2,3-triazol-4-yl)-phenol;
2,6-dibromo-5-(1H-1,2,3-triazol-4-yl)-phenol;
1H-naptho[1,2-d]-1,2,3-triazole;
2,8-dihydro-indeno[1,2-d]-1,2,3-triazole;
4-phenyl-1H-1,2,3-triazole; and
5,5a,6,8-tetrahydro-4H-acenaphtho[4,5-d]-1,2,3-triazole;
or a pharmaceutically acceptable salt or solvate thereof.

3. The method of claim 1, wherein the compound of formula (IA) is selected from:
4-(3-iodophenyl)-1H-1,2,3-triazole;
4-(2-fluorophenyl)-1H-1,2,3-triazole;
4-(2-chlorophenyl)-1H-1,2,3-triazole;
4-(3-methylphenyl)-1H-1,2,3-triazole;
4-(4-chlorophenyl)-1H-1,2,3-triazole;
4-(4-ethylphenyl)-1H-1,2,3-triazole;
4-(4-methylphenyl)-1H-1,2,3-triazole;
2-(1H-1,2,3-triazol-4-yl)-5-methylpyridine;
2-(1H-1,2,3-triazol-4-yl)-4-methyl-pyridine;
4-(thiophen-3-yl)-1H-1,2,3-triazole;
4-(4-bromophenyl)-1H-1,2,3-triazole;
4-(1,3-dichlorophenyl)-1H-1,2,3-triazole;
2-(1H-1,2,3-triazol-4-yl)-benzofuran;
furan-2-ylmethyl-(3-[1H-1,2,3-triazol-4-yl]phenyl)amine;
furan-3-ylmethyl-(3-[1H-1,2,3-triazol-4-yl]phenyl)amine;
benzyl-(3-[1H-1,2,3-triazol-4-yl]phenyl)amine;
4-(4-fluorophenyl)-1H-1,2,3-triazole;
2-bromo-5-(1H-1,2,3-triazol-4-yl)-phenol;
2,4-dibromo-5-(1H-1,2,3-triazol-4-yl)-phenol; and
2-(5-bromo-1H-1,2,3-triazol-4-yl)-4-methyl-pyridine; or a pharmaceutically acceptable salt or solvate thereof.

4. A method for treating a disease mediated by MetAP2 in mammals, comprising administering to a mammal in need of such treatment, an effective amount of a compound of formula (IA) or a pharmaceutically acceptable salt thereof: 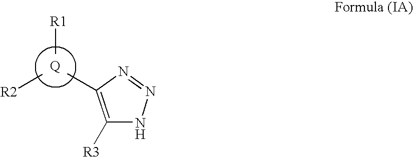
wherein:
Q is a 5- or 6-membered monocyclic ring containing up to two heteroatoms selected from N, O, or S, or an 8- to 11-membered fused bicyclic ring containing up to four heteroatoms selected from N, O, or S;
R¹ and R² are independently selected from H—, Ph-C₀₋₆alkyl-, Het-C₀₋₆ alkyl-, C₁₋₆alkyl-, C₁₋₆alkoxy-, C₁₋₆mercaptyl-, Ph-C₀₋₆alkoxy-, Het-C₀₋₆alkoxy-, HO—, R⁴R⁵N—, Het-S—C₀₋₆alkyl-, Ph-S—C₀₋₆alkyl-, HO(CH₂)₁₋₆—, R⁴R⁵N(CH₂)₂₋₆—, R⁴R⁵N(CH₂)₂₋₆O—, R⁶CO₂(CH₂)₀₋₆—, R⁶CO₂(CH₂)₂₋₆O—, R⁶SO₂(CH₂)₁₋₆—, —CF₃, —OCF₃, or halogen, and Ph or Het are substituted with up to five of C₂₋₆alkyl-, C₁₋₆alkoxy-, R⁴R⁵N(CH₂)₁₋₆—, R⁴R⁵N(CH₂)₂₋₆O—, —CO₂R⁶, —CF₃ or, halogen;
R³ is H—, halogen, or R³ and Q together form a fused bicyclic or tricyclic saturated or unsaturated fused ring system wherein R³ is —C—, or —C═C—; and
R⁴, R⁵, and R⁶ are independently selected from H—, C₂₋₆alkyl-, C₃₋₆alkenyl-, C₃₋₆alkynyl-, Ph-C₀₋₆alkyl-, Het-C₀₋₆alkyl-, or C₃₋₇cycloalkyl-C₀₋₆alkyl-.

5. The method of claim 4, wherein the compound of formula (IA) is selected from:
3-(1H-1,2,3-triazol-4-yl)-phenol;
4-(4-n-butylphenyl)-1H-1,2,3-triazole;
N-(3-[1H-1,2,3-triazol-4-yl]phenyl)benzamide;
3-(1H-1,2,3-triazol-4-yl)-phenylamine;
N-(3-[1H-1,2,3-triazol-4-yl]phenyl)acetamide;
4-(4-trifouoromethylphenyl)-1H-1,2,3-triazole;
4-(3-trifouoromethylphenyl)-1H-1,2,3-triazole;
4-(4-n-propylphenyl)-1H-1,2,3-triazole;
4-(4-methoxyphenyl)-1H-1,2,3-triazole;
2-(1H-1,2,3-triazol-4-yl)-pyridine;
4-(1H-1,2,3-triazol-4-yl)-phenylamine;
1-(1H-1,2,3-triazol-4-yl)cyclohexanol;
4-(thiophen-2-yl)-1H-1,2,3-triazole;
4-(2-methylphenyl)-1H-1,2,3-triazole;
4-(1,3-dimethylphenyl)-1H-1,2,3-triazole;
4-(1-biphenyl-2-yl)-1H-1,2,3-triazole;
4-(2-benzyloxy-phenyl)-1H-1,2,3-triazole;
2-(1H-1,2,3-triazol-4-yl)-6-methylpyridine;
3-(1H-1,2,3-triazol-4-yl)-pyridine;
4-(1H-1,2,3-triazol-4-yl)-pyridine;
4-(2-methoxyphenyl)-1H-1,2,3-triazole;
4-(2-bromophenyl)-1H-1,2,3-triazole;
4-benzo[1,3]dioxol-5-yl-1H-1,2,3-triazole;
4-benzo[1,3]dioxol-4-yl-1H-1,2,3-triazole;
4-(2-[4-chloro-phenylsulfanyl]-phenyl)-1H-1,2,3-triazole;
(3-phenyl-propyl)-(3-[1H-1,2,3-triazol-4-yl]phenyl)amine;
phenethyl-(3-[1H-1,2,3-triazol-4-yl]phenyl)amine;
napthalene-1-ylmethyl-(3-[1H-1,2,3-triazol-4-yl]phenyl)amine;
napthalene-2-ylmethyl-(3-[1H-1,2,3-triazol-4-yl]phenyl)amine;
4-(1H-1,2,3-triazol-4-yl)-phenol;
2,6-dibromo-5-(1H-1,2,3-triazol-4-yl)-phenol;
1H-naptho[1,2-d]-1,2,3-triazole;
2,8-dihydro-indeno[1,2-d]-1,2,3-triazole;
4-phenyl-1H-1,2,3-triazole; and
5,5a,6,8-tetrahydro-4H-acenaphtho[4,5-d]-1,2,3-triazole;
or a pharmaceutically acceptable salt or solvate thereof.

6. The method of claim 4, wherein the compound of formula (IA) is selected from:
4-(3-iodophenyl)-1H-1,2,3-triazole;
4-(2-fluorophenyl)-1H-1,2,3-triazole;
4-(2-chlorophenyl)-1H-1,2,3-triazole;
4-(3-methylphenyl)-1H-1,2,3-triazole;
4-(4-chlorophenyl)-1H-1,2,3-triazole;
4-(4-ethylphenyl)-1H-1,2,3-triazole;
4-(4-methylphenyl)-1H-1,2,3-triazole;
2-(1H-1,2,3-triazol-4-yl)-5-methylpyridine;
2-(1H-1,2,3-triazol-4-yl)-4-methyl-pyridine;
4-(thiophen-3-yl)-1H-1,2,3-triazole;
4-(4-bromophenyl)-1H-1,2,3-triazole;
4-(1,3-dichlorophenyl)-1H-1,2,3-triazole;
2-(1H-1,2,3-triazol-4-yl)-benzofuran;
furan-2-ylmethyl-(3-[1H-1,2,3-triazol-4-yl]phenyl)amine;
furan-3-ylmethyl-(3-[1H-1,2,3-triazol-4-yl]phenyl)amine;
benzyl-(3-[1H-1,2,3-triazol-4-yl]phenyl)amine;
4-(4-fluorophenyl)-1H-1,2,3-triazole;
2-bromo-5-(1H-1,2,3-triazol-4-yl)-phenol;
2,4-dibromo-5-(1H-1,2,3-triazol-4-yl)-phenol; and
2-(5-bromo-1H-1,2,3-triazol-4-yl)-4-methyl-pyridine; or a pharmaceutically acceptable salt or solvate thereof.

7. A compound of formula (I), or a pharmaceutically acceptable salt or solvate thereof: 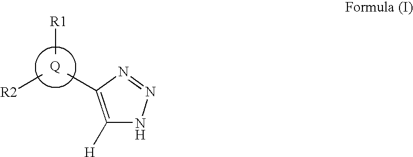
wherein:
Q is a 5- or 6-membered monocyclic ring optionally containing up to two heteroatoms selected from N, O, or S, or an 8- to 11-membered fused bicyclic ring optionally containing up to four heteroatoms selected from N, O, or S;
with the proviso that Q is substituted by up to eight of R¹; and further, if Q is phenyl (“Ph”), Q must be substituted by at least one of substituent R²;
R¹ is H—, Ph-C₀₋₆alkyl-, Het-C₀₋₆ alkyl-, C₁₋₆alkyl-, C₁₋₆alkoxy-, C₁₋₆mercaptyl-, Ph-C₀₋₆alkoxy-, Het-C₀₋₆alkoxy-, HO—, R⁴R⁵N—, Het-S—C₀₋₆alkyl-, Ph-S—C₀₋₆alkyl-, HO(CH₂)₁₋₆—, R⁴R⁵N(CH₂)₂₋₆—, R⁴R⁵N(CH₂)₂₋₆O—, R⁶CO₂(CH₂)₀₋₆—, R⁶CO₂(CH₂)₁₋₆O—, R⁶SO₂(CH₂)₁₋₆—, —CF₃, —OCP₃, or halogen, and Ph or Het are substituted with up to five of C₂₋₆alkyl-, C₁₋₆alkoxy-, R⁴R⁵N(CH₂)₁₋₆—, R⁴R⁵N(CH₂)₂₋₆O—, —CO₂R⁶, —CF₃ or, halogen;
R² is Ph-C₀₋₆alkyl-, Het-C₀₋₆ alkyl-, C₅₋₆alkyl-, C₂₋₆alkoxy-, C₁₋₆mercaptyl-, Ph-C₀₋₆alkoxy-, Het-C₀₋₆alkoxy-, HO—, R⁴R⁵N—, Het-S—C₀₋₆alkyl-, Ph-S—C₀₋₆alkyl-, HO(CH₂)₁₋₆—, R⁴R⁵N(CH₂)₂₋₆—, R⁴R⁵N(CH₂)₂₋₆O—, R⁶CO₂(CH₂)₀₋₆—, R⁶CO₂(CH₂)₁₋₆O—, R⁶SO₂(CH₂)1-6—, —CF₃ or —OCF₃, and Ph or Het are substituted with up to five of C₂₋₆alkyl-, C₁₋₆alkoxy-, R⁴R⁵N(CH₂)₁₋₆—, R⁴R⁵N(CH₂)₂₋₆O—, —CO₂R⁶, —CF₃ or, halogen;
provided that the compound of formula (I) is not [(6-(1H-1,2,3-triazol-4-yl)-2-napthalenyl)oxy]-acetic acid; [(6-(1H-1,2,3-triazol-4-yl)-2-napthalenyl)oxy]-acetic acid 1,1-dimethylethyl ester; 4-(1H-1,2,3-triazol-4-yl)-aniline; 2-chloro-4-(1H-1,2,3-triazol-4-yl)-aniline; 1-(4-fluorophenyl)-5-(1H-1,2,3-triazol-4-yl)-1H-indole; 2-(1H-1,2,3-triazol-4-yl)-pyridine; 3-(1H-1,2,3-triazol-4-yl)-pyridine; 4-(1H-1,2,3-triazol-4-yl)-phenol; 4-(2-napthyl)-1H-1,2,3-triazole; 4-[3-bromo-4-(trifluoromethoxy)phenyl]-1H-1,2,3-triazole; 4-(1H-1,2,3-triazol-4-yl)-morpholine; 5-methyl-2-(1H-1,2,3-triazol-4-yl)-1H-benzimidazole; 1(1H-1,2,3-triazol-4-yl)-1H-benzotriazole; 5-methyl-2-(1H-1,2,3-triazol-4-yl)-1H-benzotriazole; or 3-(1H-1,2,3-triazol-4-yl)-piperidine; and
R⁴, R⁵, and R⁶ are independently selected from H—, C₂₋₆alkyl-, C₃₋₆alkenyl-, C₃₋₆alkynyl-, Ph-C₀₋₆alkyl-, Het-C₀₋₆alkyl-, or C₃₋₇cycloalkyl-C₀₋₆alkyl-.

8. A pharmaceutical composition comprising a compound as claimed in claim 7 and a pharmaceutically acceptable carrier.

9. A process for making compounds of formula (IA), said process comprising:
a) carbon homologation of an aldehyde to provide a compound of formula (II) 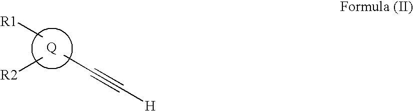
b) followed by azide cycloaddition of the compound of formula (II) to provide the compound of formula (IA), wherein Q, R¹, R² and R³ are defined as in claim 1; or alternatively,
(c) reductive amination to alkylate an aniline of formula (III) 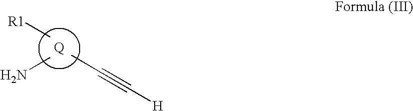
to provide a compound of formula (IV) 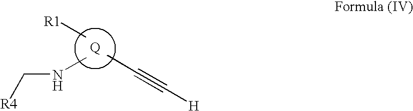
(d) followed by azide cycloaddition of the compound of formula (IV) to provide the compound of formula (IA), wherein Q, R¹, R² and R³ are defined as in claim 1.

10. A compound selected from:
4-ethynyl-benzo[1,3]dioxole;
1-(4-chloro-phenylsulfanyl)-2-ethynylbenzene;
(3-phenyl-propyl)-(3-ethynylphenyl)amine;
phenethyl-(3-ethynylphenyl)-amine;
furan-2-ylmethyl-(3-ethynylphenyl)-amine;
furan-3-ylmethyl-(3-ethynylphenyl)-amine;
napthalene-1-ylmethyl-(3-ethynylphenyl)-amine; and
napthalene-2-ylmethyl-(3-ethynylphenyl)-amine.
